(19) United States
(12) Patent Application Publication
(10) Pub. No.: US 2017/0274087 A1
(43) Pub. Date: Sep. 28, 2017

(54) SACCHARIDE ANALOGS AND AGENTS FOR THE DIAGNOSIS AND THERAPY OF BACTERIAL INFECTIONS

(71) Applicants: Emory University, Atlanta, GA (US); The Regents of the University of California, Oakland, CA (US); Georgia Tech Research Corporation, Atlanta, GA (US)

(72) Inventors: Mark Goodman, Atlanta, GA (US); Robert W. Taylor, Stone Mountain, GA (US); Kiyoko Takemiya, Decatur, GA (US); Niren Murthy, Berkeley, CA (US); Rafi Mohammed, Berkeley, CA (US); Xinghai Ning, Albany, CA (US)

(21) Appl. No.: 15/512,696

(22) PCT Filed: Sep. 21, 2015
(86) PCT No.: PCT/US2015/051262

§ 371 Date: Mar. 20, 2017

Related U.S. Application Data
(60) Provisional application No. 62/052,545, filed on Sep. 19, 2014.
(60) Provisional application No. 62/068,984, filed on Oct. 27, 2014.

(51) Int. Cl. A61K 51/06; A61K 51/04; A61K 49/00; C07H 15/06; C08B 37/00

(52) U.S. Cl. CPC A61K 47/48092; A61K 51/0491; A61K 51/06; A61K 49/0052; A61K 49/0054; A61K 47/4823

(57) ABSTRACT

This disclosure relates saccharide analogs such as thiomaltose-based analogs for targeting bacteria and related uses. In certain embodiments, the disclosure relates to methods of transferring a molecule of interest into bacteria comprising mixing bacteria with a non-naturally occurring conjugate, wherein the conjugate comprises a thiomaltose-based analog and a molecule of interest under conditions such that the conjugate is transported across the bacterial cell wall. In certain embodiments, the molecule of interest can be a tracer or an antibiotic.

### CROSS REFERENCE TO RELATED APPLICATIONS

This application claims the benefit of priority to U.S. Provisional Applications 62/052,545, filed on Sep. 19, 2014, and 62/068,984, filed on Oct. 27, 2014, the contents of which are hereby incorporated in their entirety.

### BACKGROUND

The diagnosis and therapy of bacterial infections remains a central challenge in medicine. Infections are currently diagnosed by using blood cultures or tissue biopsy; however, these methods can only detect late stage infections that are challenging to treat, and also cannot detect bacterial drug resistance. A major limitation preventing the effective treatment of bacterial infection is an inability to image infections in vivo with accuracy and sensitivity. Consequently, bacterial infections can be diagnosed only after they have become systematic or have caused significant anatomical tissue damage, a stage at which they are challenging to treat owing to the high bacterial burden. Although contrast agents have been developed to image bacteria, their clinical impact has been minimal because they are unable to detect small numbers of bacteria in vivo and cannot distinguish infections from other pathologies such as cancer and inflammation. There is a need for the development of contrast agents that can image small numbers of bacteria accurately in vivo.

Bacteria can utilize glycogen, starch, and amylose as carbon sources. Prior to transport through the cell membrane, these polysaccharides are hydrolyzed by the extracellular α-amylase into smaller maltodextrins, maltose and isomaltose. The maltose ABC importer (type I) of Escherichia coli enables the bacteria to feed on maltose and maltodextrins (Bordignon et al., Mol Microbiol., 2010, 77(6):1354-1366). Although, maltohexaose contrast agents have been developed to image bacteria, they are hydrolyzed by the serum amylase. There is a great need for the development of more stable targeting agents that can diagnose and treat the bacterial infections.

Murthy et al. report oligosaccharides conjugates for targeting bacteria. See WO/2012/097223.

Hindsgaul reports the preparation of thio galactosides as toxin inhibitor bactericides, virucides, and fungicides. U.S. Pat. No. 5,932,554

Zeng et al. report a process for selective removal of saccharide thioacetyl protective group. CN Patent 103554195.

Gottschaldt et al. report the synthesis of sugar-substituted polypyridine metal complexes and their use as diagnostic, visualization, or therapeutic agents in the treatment of disease. DE Patent 102007032799.

References cited herein are not an admission of prior art.

### SUMMARY

This disclosure relates saccharide analogs such as thiomaltose-based analogs for targeting bacteria and related uses. In certain embodiments, the disclosure relates to methods of transferring a molecule of interest into bacteria comprising mixing bacteria with a non-naturally occurring conjugate, wherein the conjugate comprises a thiomaltose-based analog and a molecule of interest under conditions such that the conjugate is transported across the bacterial cell wall. In certain embodiments, the molecule of interest can be a tracer or an antibiotic.

In certain embodiments, the disclosure relates to a composition comprising a tracer molecule conjugated to thiol bridging saccharides. In certain embodiments the tracer molecule is a positron-emitting radionuclide. In certain embodiments, the positron-emitting radionuclide is selected from carbon-11, nitrogen-13, oxygen-15, fluorine-18, rubidium-82, and strontium-82. In certain embodiments the tracer is a fluorescent molecule. In certain embodiments, the fluorescent molecule is a fluorescent dye. In certain embodiments, the thiol bridging saccharide is thiomaltose-perylene.

With regard to any of the conjugates disclosed herein, the saccharides can be a polysaccharide of greater than 2, 3, 4, 5, or 6 sugar oligomers bridged by one or more thiol linkages which are typically isolated or substantially purified. In some embodiments, the polysaccharide comprises glucose oligomers, e.g., maltohexaose, a polysaccharide with 6 glucose oligomers. Typically, the glucose oligomers are linked by a thiol bridging alpha 1→4, i.e., 1 to 4, covalent bond. In certain embodiments, the disclosure contemplates thiomaltose-based analog of glucose oligomers and/or 2-deoxyglucose oligomers wherein one or more of the glucose monomers are substituted with a positron-emitting radionuclide ¹⁸F.

In certain embodiments, any of the compounds or saccharide analogs disclosed herein or derivatives can be optionally substituted with one or more, the same or different, substituents.

In certain embodiments, the disclosure relates to an antibiotic conjugated to a saccharide analog disclosed herein. In certain embodiments, the antibiotic is selected from the group comprising sulfonamides, carbapenems, penicillins, diaminopyrimidines, quinolones, beta-lactam antibiotics, cephalosporins, tetracyclines, notribenzenes, aminoglycosides, macrolide antibiotics, polypeptide antibiotics, nitrofurans, nitroimidazoles, nicotinin acids, polyene antibiotics, imidazoles, glycopeptides, cyclic lipopeptides, glycylcyclines, and oxazolidinones. In certain embodiments, the antibiotic is selected from dapsone, paraaminosalicyclic, sulfanilamide, sulfamethizole, sulfamethoxazole, sulfapyridine, trimethoprim, pyrimethamine, nalidixic acid, norfloxacin, ciproflaxin, cinoxacin, enoxacin, gatifloxacin, gemifloxacin, grepafloxacin, levofloxacin, lomefloxacin, moxifloxacin, ofloxacin, pefloxacin, sparfloxacin, trovafloxacin, amoxicillin, ampicillin, azlocillin, carbenicillin, cloxacillin, dicloxacillin, flucloxacillin, hetacillin, oxacillin, mezlocillin, penicillin G, penicillin V, piperacillin, cefacetrile, cefadroxil, cefalexin, cefaloglycin, cefaloniumn, cefaloridin, cefalotin, cefapirin, cefatrizine, cefazaflur, cefazedone, cefazolin, cefradine, cefroxadine, ceftezole, cefaclor, cefonicid, ceforanide, cefprozil, cefuroxime, cefuzonam, cefmetazole, cefoteta, cefoxitin, cefcapene, cefdaloxime, cefdinir, cefditoren, cefetamet, cefixime, cefmenoxirme, cefodizime, cetbperazone, cefotaxime, cefotiam, cefpimizole, cefpiramide, cefpodoxime, cefteram, ceftibuten, ceftiofur, ceftiolen, ceftizoxime, ceftriaxone, cefoperazone, ceftazidime, cefepime, moxolactam, imipenem, ertapenem, meropenem, aztreonam, oxytetracycline, chlortetracycline, clomocycline, demeclocycline, tetracycline, doxycycline, lymecycline, meclocycline, methacycline, minocycline, rolitetracycline, chloramphenicol, amikacin, gentamicin, farmycetin, kanamycin neomicin, neomycin, netilmicin, streptomycin, tobramycin, azithromycin, clarithromycin, dirithromycin, erythromycin, roxithromycin, telithromycin, polymyxin-B, colistin, bacitracin, tyrothricin, notrifurantoin, furazolidone, metronidazole, tinidazole, isoniazid, pyrazinamide, ethionamide, nystatin, amphotericin-B, hamycin, miconazole, clotrimazole, ketoconazole, fluconazole, rifampacin, lincomycin, clindamycin, spectinomycin, chloramphenicol, clindamycin, colistin, fosfomycin, loracarbef, nitrofurantoin, procain, spectinomycin, tinidazole, ramoplanin, teicoplanin, and vancomycin.

In certain embodiments, the disclosure relates to a method of transferring a molecule of interest into bacteria comprising mixing bacteria with a non-naturally occurring conjugate under conditions such that the conjugate is transported across the bacterial cell wall wherein the conjugate comprises a saccharide analog and a molecule of interest.

In certain embodiments, the disclosure relates to an imaging method comprising a) administering a tracer molecule conjugated to a saccharide analog to a subject; and b) scanning the subject for a physical property of the tracer molecule. In certain embodiments the imaging method further comprises the step of detecting the physical property of the tracer molecule and creating an image highlighting the location of the tracer molecule in the subject.

In certain embodiments, the disclosure relates to a method of treating or preventing a bacterial infection comprising administering an effective amount of an isolated conjugate to a subject in need thereof wherein the isolated conjugate comprises an antibiotic and a saccharide analog. In certain embodiments the isolated conjugate is administered in combination with another antibiotic.

### BRIEF DESCRIPTION OF THE DRAWINGS

FIG. 1 illustrates saccharide analogs, e.g., thiomaltose-based analogs designed to image bacterial infections. Left side: illustrates bacteria internalized PET contrast agent (1) with an ¹⁸F derivatized maltodextrin probe (MDP) (1) through the maltodextrin transporter (2). ¹⁸F-thiomaltose is designed to image bacteria in vivo by targeting the maltodextrin transport pathway. ¹⁸F-thiomaltose is internalized by bacteria. Maltodextrin transporters are not present in mammalian cells and ¹⁸F-thiomaltose-based analogs therefore have high specificity for bacteria over mammalian cells. Right side: illustrates imaging bacterial infections in implanted device due to robust accumulation of ¹⁸F-MDPs. Systemic injection (4) of ¹⁸F-MDPs can be used to image bacterial implant infections and generate an imaging agent that can diagnose early stage bacterial implant infections at the site of implant (3).

FIG. 2 Synthesis of thiomaltose-perylene: a) NaOCH₃, MeOH, 55%. b) Tf₂O, DCM-Pyridine, 63%. c) TEA, DMF, 38%. d) NaOCH₃, MeOH, 83%. e) Pyridine, Ac₂O, 78%. f) NH₂NH₂.HOAc, DMF, 81%. g) Trichloroacetonitrile, DBU, DCM, 97%. h) Azidopropanol, TMSOTf, DCM, 63%. i) 1. alkyne-perylene, CuI, DIPEA, DMF; 2. LiOH, MeOH, H2O, 65% in two steps

FIG. 3 Synthesis of ¹⁸F-thiomaltose: a) 2, CuI, DIPEA, DMF. b) 1. K¹⁸F, CH₃CN, 110° C.; 2. NaOH, H₂O.

FIG. 4 schematically illustrates the retrosynthesis of thiomaltose.

FIG. 5 schematically illustrates the synthesis of the glucose building block.

FIG. 6 schematically illustrates the synthesis of the galactose building block.

FIG. 7 schematically illustrates the synthesis of the galactose building block.

FIG. 8 schematically illustrates the synthesis of the galactose building block.

FIG. 9 schematically illustrates glycosylation.

FIG. 10 schematically illustrates the synthesis of perylene-thiomaltose.

FIG. 11 shows uptake of thiomaltose-perylene in Mtb cells. Thiomaltose-perylene (50 μM), and uptake was measured using a plate reader (fluorescence intensity at 410/480).

FIG. 12 shows a table of uptake of thiomaltose-perylene in Salmonella and pseudomonas cells.

FIG. 13 shows uptake studies in bacterial and mammalian cells. Bacteria (E. coli) and macrophages were incubated with thiomaltose-perylene (20 μM) for 2 hours. Cells were washed 3 times with PBS. Cells were lysed using they lysis buffer. The fluorescence intensity from bacteria and mammalian cells was measured by plate reader at 410/480 and the background of lysis buffer was subtracted. The protein content was analyzed by BCA assay. The uptake of thiomaltose-perylene in bacteria was 98 fold higher than in macrophages.

FIG. 14 shows data indicating that thiomaltose is resistant to maltase hydrolysis. Maltose is hydrolyzed completely in 3 hours, whereas thiomaltose is less than 1% hydrolyzed.

FIG. 15 illustrates certain embodiments of the disclosure.

FIG. 16 illustrates certain embodiments of the disclosure.

FIG. 17 illustrates certain embodiments of the disclosure.

FIG. 18 illustrates certain embodiments of the disclosure.

FIG. 19 shows data indicating that the thiomaltose-radezolid inhibits bacterial growth. E. Coli were grown in a 96 well plate, starting at an O.D. of 0.05. Thiomaltose-radezolid was added to the bacteria, and the bacteria were shaken at 37 C between 1-24 hours. The O.D. of the bacteria was then measured and normalized to untreated bacteria IC₅₀ at 24 hours is approximately 0.5 micromolar.

FIG. 20 illustrates certain embodiments of the disclosure.

FIG. 21 illustrates certain embodiments of the disclosure.

FIG. 22 illustrates certain embodiments of the disclosure.

FIG. 23 shows data indicating TM-R can effectively kill P. aeruginosa. TM-R and free radezolid were incubated with P. aeruginosa for 24 hours. OD600 were used to determine the bacteria growth.

FIG. 24 illustrates certain embodiments of the disclosure.

FIG. 25 illustrates certain embodiments of the disclosure.

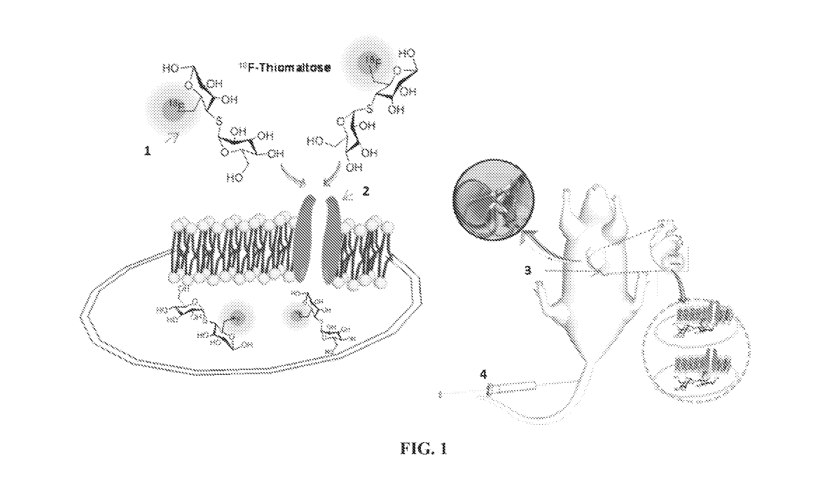

### DETAILED DESCRIPTION

Before the present disclosure is described in greater detail, it is to be understood that this disclosure is not limited to particular embodiments described, and as such can, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

### Targeting Bacteria

A central problem in imaging bacterial infections is to develop targeting strategies that can deliver large quantities of imaging probes to bacteria. This has been challenging because typical imaging probes target the bacterial cell wall and cannot access the bacterial intracellular volume. Although numerous contrast agents have been developed to image bacteria, their clinical impact has been minimal because they are unable to detect small numbers of bacteria in vivo, and cannot distinguish infections from other pathologies such as cancer and inflammation. Within certain embodiments, the disclosure relates to a thiomaltose-based imaging probe which can detect bacteria in vivo with a sensitivity two orders of magnitude higher than previously reported, and can detect bacteria using a bacteria-specific mechanism that is independent of host response and secondary pathologies.

In certain embodiments, the thiomaltose-based imaging probe is composed of a fluorescent dye conjugated to a thiomaltose-based analog and is rapidly internalized through the bacteria-specific maltodextrin transport pathway, endowing the thiomaltose-based imaging probes with a unique combination of high sensitivity and specificity for bacteria. Certain thiomaltose-based imaging probes selectively accumulate within bacteria at millimolar concentrations, and are a thousand-fold more specific for bacteria than mammalian cells. Furthermore, thiomaltose-based imaging probes can image as few as 10⁵ colony-forming units in vivo and can discriminate between active bacteria and inflammation induced by either lipopolysaccharides or metabolically inactive bacteria.

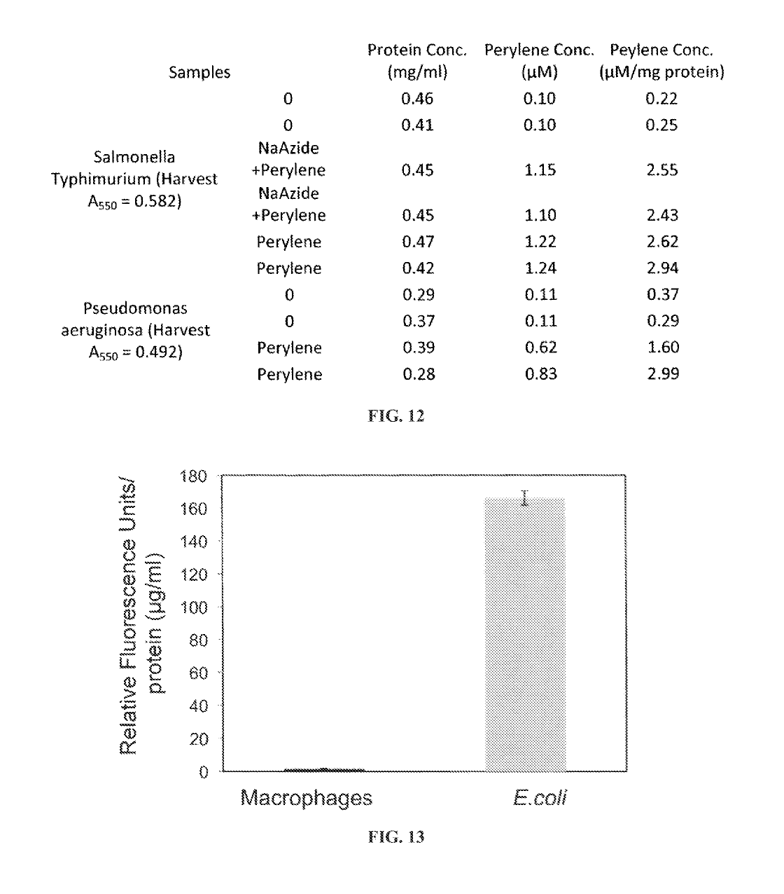

Contrast agents that are robustly internalized through the bacteria-specific maltodextrin transporter and can image bacterial infections in vivo with improved sensitivity and specificity (see FIGS. 11-14). Thiomaltose-based imaging probes can deliver millimolar concentrations of imaging probes into bacteria, making it possible to image low numbers of bacteria. Thiomaltose-based imaging probes also have high specificity for bacteria because mammalian cells do not express the maltodextrin transporter and cannot internalize contrast agents conjugated to thiomaltose-based analogs (FIG. 13). Thiomaltose-based imaging probes are typically composed of α (1→4)-thiol linked glucose oligomers. Because thiomaltose-based imaging probes are typically hydrophilic and membrane impermeable, they are efficiently cleared from uninfected tissues in vivo, leading to a low background. Furthermore, the lumen of intestinal tissues or the outer layers of the skin are not permeable to glucose oligomers. Thiomaltose-based imaging probes delivered systemically should therefore not be internalized by the resident bacterial microflora present in healthy subjects.

### Conjugates, Derivatives, and Related Compounds

In certain embodiments, the disclosure relates to compounds of formula I,

Q-E-G Formula I

or salts thereof wherein,

Q is a polysaccharide comprising a 1 to 4-thiol or oxygen linkage such as thiomaltose;

E is a linking group; and

G is a tracer, a drug, an antibiotic, an azide group, or other molecule of interest.

In certain embodiments, Q is a thiomaltose-based analog comprising glucose, a glucose

derivative, and/or a substituted glucose oligomer.

In certain embodiments, E contains a triazole positioned between linking groups such as the

following groups alone or in combination, methylene, ethylene, ether, amine, amide, ester, carbonyl, thiol, dithiol, thiolester, aromatic, heteroaromatic, or saturated or unsaturated hydrocarbon groups.

In certain embodiments, E can be represented by a formula:

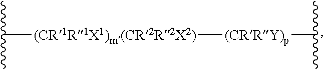

wherein the symbol [symbol] represents the point of attachment to Q and G;

m′ is 0, 1, 2, 3, 4, 5, 6, 7, 8, 9, 10, 11, 12, 13, 14, 15, 16, 17, 18, 19, 20, 21, 22, or 23;

p is 0, 1, 2, 3, 4, 5, 6, 7, 8, 9, 10, 11, 12, 13, 14, 15, 16, 17, 18, 19, 20, 21, 22, or 23;

R′¹, R″¹, R′² and R″² are at each occurrence individually and independently hydrogen, halogen, alkyl, alkoxy, or hydroxyl;

X¹, X² and Y is each occurrence individually and independently —O—, —S—, —S—S—, —NH—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH2CH2O)_{q}—, —(CH₂)ᵣ—, a disubstituted carbocyclyl, a di-substituted aryl, a disubstituted heterocyclyl, or absent;

q can be 1 to 1000;

r can be 1 to 22.

In certain embodiments, E can be connected to Q via the the anomeric carbon at the reducing end of the polysaccharide

In certain embodiments, the disclosure relates to compounds of formula I with Formula IA

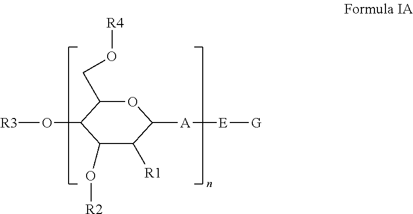

or salts thereof wherein,

A is independently O or S at each occurrence;

n is 2, 3, 4, 5, 6, 7, 8, 9, 10, 11, 12, 13, 14, or 15;

E is a linking group, for instance as defined above;

G is a molecule of interest such as a radionuclide, fluorescent moiety, an antibiotic, or an azide group;

R1, R2, R3, and R4, are each individually and independently a protecting group, hydrogen, alkyl, halogen, nitro, cyano, hydroxy, amino, mercapto, formyl, carboxy, alkanoyl, carbamoyl, alkoxy, alkylthio, alkylamino, (alkyl)2amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alky)₂amino, alkylsulfinyl, alkylsulfonyl, arylsulfonyl, carbocyclyl, aryl, or heterocyclyl, wherein R5 is optionally substituted with one or more, the same or different, R6; and

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, A must be S at least once or all of A are S.

In certain embodiments, n is 5 or 6 or more, or n is 3 or 4 or more.

In certain embodiments, R2, R3, and R4 are hydrogen or alkanoyl optionally substituted with R5.

In certain embodiments, R1 is hydrogen, halogen, or hydroxy substituted with a protecting group.

In certain embodiments, R1 is ¹⁸F.

In certain embodiments, E is triazole positioned between linking groups such as the following groups alone or in combination, ether, amine, amide, ester, carbonyl, thiol, dithiol, thiolester, aromatic, heteroaromatic, or hydrocarbon groups.

In certain embodiments, G is ¹⁸F.

Certain embodiments include compounds of Formula IA-1:

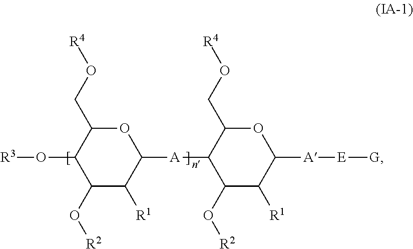

Wherein A is either S or O, and A′ is O, provided that at least one A is S.

In certain embodiments, n′ 1 or 2, n′ is 5 or 6 or more, or n′ is 3 or 4 or more.

In certain embodiments, R², R³, and R⁴ are hydrogen or alkanoyl optionally substituted with R5.

In certain embodiments, R¹ is hydrogen, halogen, or hydroxyl, optionally substituted with a protecting group.

In certain embodiments, R¹ is ¹⁸F.

E is a linker, and in certain embodiments, E can be represented by a formula:

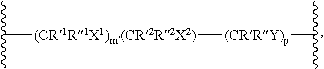

wherein the symbol [symbol] represents the point of attachment to A′ and G;

m′ is 0, 1, 2, 3, 4, 5, 6, 7, 8, 9, 10, 11, 12, 13, 14, 15, 16, 17, 18, 19, 20, 21, 22, or 23;

p is 0, 1, 2, 3, 4, 5, 6, 7, 8, 9, 10, 11, 12, 13, 14, 15, 16, 17, 18, 19, 20, 21, 22, or 23;

R′¹, R″¹, R′² and R″² are at each occurrence individually and independently hydrogen, halogen, alkyl, alkoxy, or hydroxyl;

X¹, X² and Y is each occurrence individually and independently —O—, —S—, —S—S—, —NH—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH2CH2O)_{q}—, —(CH₂)ᵣ—, a disubstituted carbocyclyl, a di-substituted aryl, a disubstituted heterocyclyl, or absent;

q can be 1 to 1000;

r can be 1 to 22.

In certain embodiments, E is triazole positioned between linking groups such as the following groups alone or in combination, ether, amine, amide, ester, carbonyl, thiol, dithiol, thiolester, aromatic, heteroaromatic, or hydrocarbon groups.

In certain embodiments, G is ¹⁸F.

Certain embodiments include thioglycoside compounds of Formula IA-2:

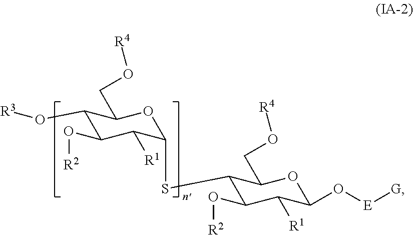

Wherein R¹-R⁴, n′, E and G are as defined above. In certain embodiments, R², R³ and R⁴ are each hydrogen, and R¹ is either hydroxyl, hydrogen or halogen, preferably ¹⁸F.

In certain embodiments, the disclosure relates to compounds of formula I with formula IB

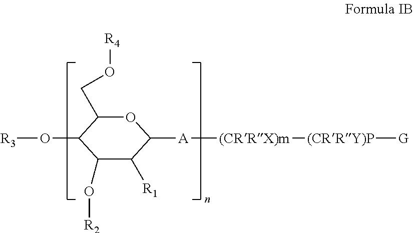

or salts thereof wherein,

A is O or S at each occurrence provided A must be S at least once or all of A are S;

n is 2, 3, 4, 5, 6, 7, 8, 9, 10, 11, 12, 13, 14, or 15;

m is 1, 2, 3, 4, 5, 6, 7, 8, 9, 10, 11, 12, 13, 14, 15, 16, 17, 18, 19, 20, 21, 22, 23, or 24;

p is 0, 1, 2, 3, 4, 5, 6, 7, 8, 9, 10, 11, 12, 13, 14, 15, 16, 17, 18, 19, 20, 21, 22, or 23;

R′ and R″ are at each occurrence individually and independently hydrogen, halogen, alkyl, alkoxy, or hydroxyl;

X and Y are at each occurrence individually and independently —O—, —S—, —S—S—, —NH—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH2CH2O)_{q}—, —(CH₂)ᵣ—, a disubstituted carbocyclyl, a di-substituted aryl, a disubstituted heterocyclyl, or absent;

q can be 1 to 1000;

r can be 1 to 22;

G is a radionuclide, fluorescent molecule, an antibiotic, or an azide group;

R1, R2, R3, and R4, are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6; and

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, thylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl,

methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, X or Y is a di-substituted 1,2,3-triazole.

In certain embodiments, the compound of Formula IB can be represented by the compound of Formula IB-1:

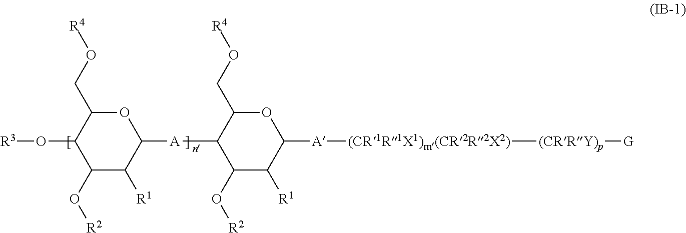

or salts thereof wherein,

A is O or S at each occurrence provided A must be S at least once or all of A are S;

A′ is O;

n′ is 1, 2, 3, 4, 5, 6, 7, 8, 9, 10, 11, 12, 13, or 14;

m′ is 0, 1, 2, 3, 4, 5, 6, 7, 8, 9, 10, 11, 12, 13, 14, 15, 16, 17, 18, 19, 20, 21, 22, or 23;

p is 0, 1, 2, 3, 4, 5, 6, 7, 8, 9, 10, 11, 12, 13, 14, 15, 16, 17, 18, 19, 20, 21, 22, or 23;

R′¹, R″¹, R′² and R″² are at each occurrence individually and independently hydrogen, halogen, alkyl, alkoxy, or hydroxyl;

X¹ is in each occurrence absent;

X² and Y are at each occurrence individually and independently —O—, —S—, —S—S—, —NH—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH2CH2O)—, —(CH₂)—, a disubstituted carbocyclyl, a di-substituted aryl, a disubstituted heterocyclyl, or absent;

q can be 1 to 1000;

r can be 1 to 22;

G is a radionuclide, fluorescent molecule, an antibiotic, or an azide group;

R1, R2, R3, and R4, are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6; and

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, thylamino, dimethylamino, diethylamino, N-methyl-N-ethyl amino, acetylamino, Nmethylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl,

methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, X² is a di-substituted 1,2,3-triazole, in other embodiments, Y is absent, and in certain preferred embodiments, X² is a di-substituted 1,2,3-triazole and Y is absent.

In some embodiments, G can be the following:

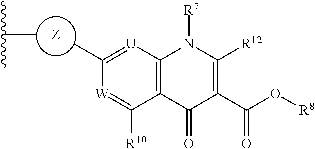

wherein the symbol [symbol] represents the point of attachment to E;

U is N or CR¹¹;

W is N or CR⁹;

Z is a carbocyclic or heterocyclic ring;

R⁷ is alkyl, carbocyclyl, or aryl, wherein R⁷ is optionally substituted with one or more R¹³; or R⁷ and R¹¹ form a heterocarbocyclic ring optionally substituted with R¹³;

R⁸ is hydrogen, alkyl or alkanoyl;

R⁹ is a hydrogen or halogen;

R¹⁰ is hydrogen, alkoxy, amino, or alkyl;

R¹¹ is hydrogen, alkoxy, or halogen; and

R¹² is hydrogen;

R¹³ is in each occurrence independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethyl amino, N-methyl-N-ethylamino, acetylamino, Nmethylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl. In certain preferred embodiments, R7 is cyclopropyl or phenyl, optionally substituted by one or more fluorine atoms.

In certain embodiments, Z can have the formula:

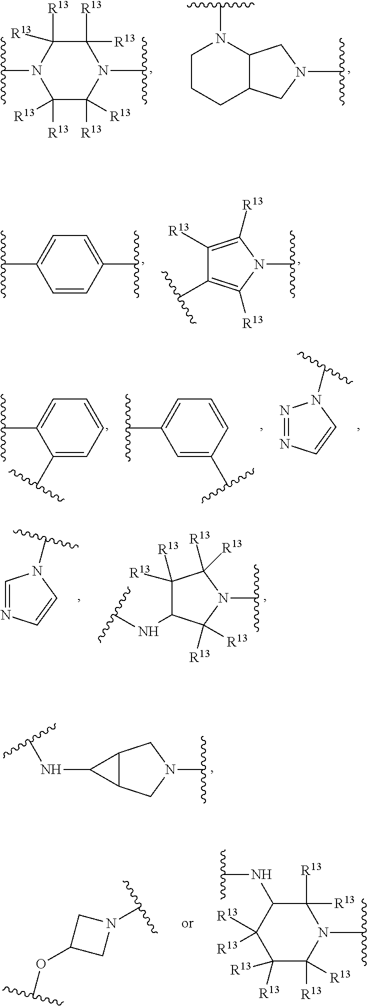

wherein the symbol [symbol] represents the point of attachment to E or or the quinolone fragment and R¹³ is as defined above. In preferred embodiments, R¹³ is in each case independently selected from hydrogen, methyl, or ethyl.

In some embodiments, G can be the following:

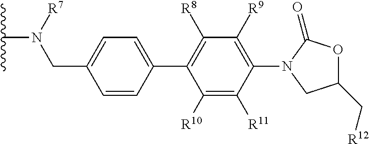

wherein the symbol [symbol] represents the point of attachment to E;

R⁷ is hydrogen, alkyl, carbocyclyl, or aryl, wherein R⁷ is optionally substituted with one or

more, the same or different R¹³;

R⁸, R⁹, R¹⁰, and R¹¹ are each individually and independently hydrogen, alkyl, halogen, nitro, cyano, hydroxy, amino, mercapto, formyl, carboxy, alkanoyl carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R⁸, R⁹, R¹⁰, and R¹¹ are optionally substituted with one or more, the same or different, R¹³;

R¹² is acetylamino, hydrogen, alkyl, halogen, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl optionally substituted with one or more, the same or different, R¹³;

R¹³ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

It certain preferred embodiments, R¹² is acetylamino.

In certain embodiments, G can be:

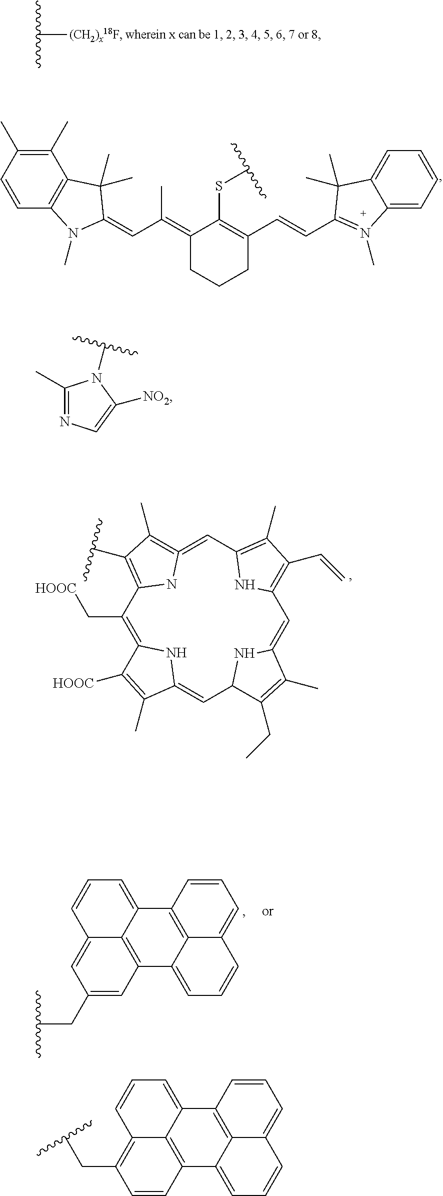

wherein the symbol [symbol] represents the point of attachment to E.

For embodiments in which G is —(CH₂)ₓ¹⁸F, it is preferred that p is 0.

In certain embodiments, the disclosure relates to compounds of formula I with formula IC,

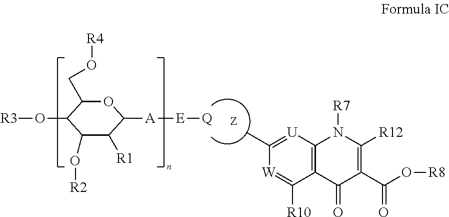

or salts thereof wherein,

n is 2, 3, 4, 5, 6, 7, 8, 9, 10, 11, 12, 13, 14, or 15;

A is O or S at each occurrence provided A must be S at least once or all of A are S;

E is a linking group;

Q is N in the ring of Z, or N is an amino or alkylamino group attached to the Z ring; or Q is O of an oxygen attached to the Z ring, wherein the Z ring can be optionally substituted with one or more, the same or different, R13;

U is N or CR11;

W is N or CR9;

Z is a carbocyclic or heterocyclic ring;

R1, R2, R3, and R4, are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6;

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methyl amino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R7 is alkyl, carbocyclyl, or aryl, wherein R7 is optionally substituted with one or more, the same or different R13; or R7 and R11 form a heterocarbocyclic ring optionally substituted

with R13;

R8 is hydrogen, alkyl or alkanoyl;

R9 is a hydrogen or halogen;

R10 is hydrogen, alkoxy, amino, or alkyl;

R11 is hydrogen, alkoxy, or halogen; and

R12 is hydrogen;

R13 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methyl amino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, E is —(CR′R″X)ₘ—(CR′R″Y)ₚ— wherein

m is 1, 2, 3, 4, 5, 6, 7, 8, 9, 10, 11, 12, 13, 14, 15, 16, 17, 18, 19, 20, 21, 22, 23, or 24;

p is 0, 1, 2, 3, 4, 5, 6, 7, 8, 9, 10, 11, 12, 13, 14, 15, 16, 17, 18, 19, 20, 21, 22, or 23;

R′ and R″ are at each occurrence individually and independently hydrogen, alkyl, halogen, or hydroxyl;

X and Y are at each occurrence individually and independently —O—, —S—, —S—S—, —NH—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH₂CH₂O)_{q}—, —(CH₂)ᵣ—, a disubstituted carbocyclyl, a di-substituted aryl, a disubstituted heterocyclyl, or absent;

q can be 1 to 1000; and

r can be 1 to 22.

In certain embodiments, the disclosure relates to compounds of formula I with formula ID,

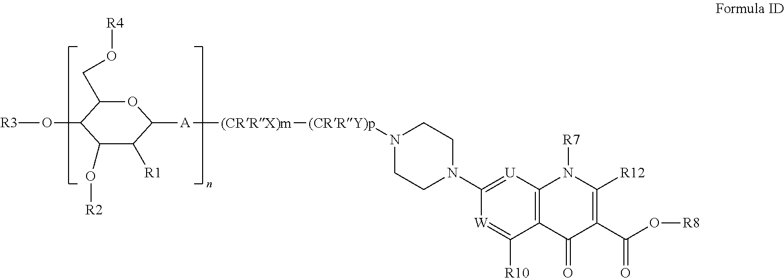

or salts thereof wherein,

U is N or CR11;

W is N or CR9;

A is O or S at each occurrence provided A must be S at least once or all of A are S;

n is 2, 3, 4, 5, 6, 7, 8, 9, 10, 11, 12, 13, 14, or 15;

m is 1, 2, 3, 4, 5, 6, 7, 8, 9, 10, 11, 12, 13, 14, 15, 16, 17, 18, 19, 20, 21, 22, 23, or 24;

p is 0, 1, 2, 3, 4, 5, 6, 7, 8, 9, 10, 11, 12, 13, 14, 15, 16, 17, 18, 19, 20, 21, 22, or 23;

R′ and R″ are at each occurrence individually and independently hydrogen, alkyl, halogen, or hydroxyl;

X and Y are at each occurrence individually and independently —O—, —S—, —S—S—, —NH—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH₂CH₂O)_{q}—, —(CH₂)ᵣ—, a disubstituted carbocyclyl, a di-substituted aryl, a disubstituted heterocyclyl, or absent;

q can be 1 to 1000;

r can be 1 to 22;

R1, R2, R3, and R4, are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6;

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-N-methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R7 is alkyl, carbocyclyl, or aryl, wherein R7 is optionally substituted with one or more, the same or different R13; or R7 and R11 form a heterocarbocyclic ring optionally substituted with R13;

R8 is hydrogen, alkyl or alkanoyl;

R9 is a hydrogen or halogen;

R10 is hydrogen, alkoxy, amino, or alkyl;

R11 is hydrogen, alkoxy, or halogen; and

R12 is hydrogen;

R13 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methyl amino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the disclosure relates to compounds of formula I with formula IF,

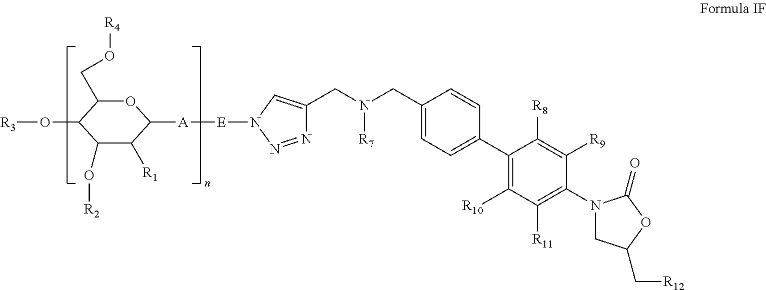

or salts thereof wherein,

n is 2, 3, 4, 5, 6, 7, 8, 9, 10, 11, 12, 13, 14, or 15;

A is O or S at each occurrence provided A must be S at least once or all of A are S;

E is a linking group; for example, of formula

In certain embodiments, E can be represented by a formula:

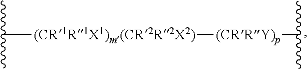

wherein the symbol [symbol] represents the point of attachment to A and the triazole

m′ is 0, 1, 2, 3, 4, 5, 6, 7, 8, 9, 10, 11, 12, 13, 14, 15, 16, 17, 18, 19, 20, 21, 22, or 23;

p is 0, 1, 2, 3, 4, 5, 6, 7, 8, 9, 10, 11, 12, 13, 14, 15, 16, 17, 1718, 19, 20, 21, 22, or 23;

R′¹, R″¹, R′² and R″² are at each occurrence individually and independently hydrogen, halogen, alkyl, alkoxy, or hydroxyl;

X¹ is each occurrence individually and independently —O—, —S—, —S—S—, —NH—, —(C═O)—, —NH(C═O)—, (C═O)NH— —O(C═O)—, —(C═)O—, —S(C═O)—, —(C═O)S—, —SO—, —SO₂, —NHSO₂—, —SO₂NH—, —(CH2CH2O)_{q}—, —(CH₂)ᵣ—, a disubstituted carbocyclyl, a di-substituted aryl, a disubstituted heterocyclyl, or absent;

X² and Y are at each occurrence individually and independently —O—, —S—, —S—S—, —NH—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH2CH2O)_{q}, —(CH₂)ᵣ—, a disubstituted carbocyclyl, a di-substituted aryl, a disubstituted heterocyclyl, or absent;

q can be 1 to 1000;

r can be 1 to 22;

R1, R2, R3, and R4, are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfinyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6;

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methyl amino, ethylamino, dimethylamino, diethylamino N-methyl-N-ethylamino, acetylamino, Nmethylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R7 is hydrogen, alkyl, carbocyclyl, or aryl, wherein R7 is optionally substituted with one or

more, the same or different R13;

R8, R9, R10, and R11 are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R8, R9, R10, and R11 are optionally substituted with one or more, the same or different, R13;

R12 is acetylamino, hydrogen, alkyl, halogen, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl optionally substituted with one or more, the same or different, R13;

R13 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, E is —(CR′R″X)ₘ—(CR′R″Y)ₚ— wherein

m is 1, 2, 3, 4, 5, 6, 7, 8, 9, 10, 11, 12, 13, 14, 15, 16, 17, 18, 19, 20, 21, 22, 23, or 24;

p is 0, 1, 2, 3, 4, 5, 6, 7, 8, 9, 10, 11, 12, 13, 14, 15, 16, 17, 18, 19, 20, 21, 22, or 23;

In certain embodiments, E is —(CH2)ₘ— wherein m is is 1, 2, or 3.

R′ and R″ are at each occurrence individually and independently hydrogen, alkyl, halogen, or hydroxyl;

X and Y are at each occurrence individually and independently —O—, —S—, —S—S—, —N—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH₂CH₂O)_{q}—, —(CH₂)—, a disubstituted carbocyclyl, a di-substituted aryl, a disubstituted heterocyclyl, or absent;

q can be 1 to 1000; and

r can be 1 to 22.

In certain embodiments, the disclosure relates to compounds of formula I with formula IG,

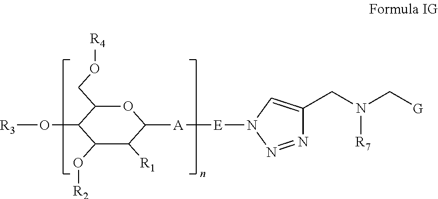

or salts thereof wherein,

n is 2, 3, 4, 5, 6, 7, 8, 9, 10, 11, 12, 13, 14, or 15;

A is O or S at each occurrence provided A must be S at least once or all of A are S;

E is a linking group; for example a formula:

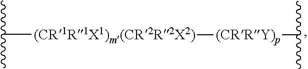

wherein the symbol [symbol] represents the point of attachment to A and triazole;

m′ is 0, 1, 2, 3, 4, 5, 6, 7, 8, 9, 10, 11, 12, 13, 14, 15, 16, 17, 18, 19, 20, 21, 22, or 23;

p is 0, 1, 2, 3, 4, 5, 6, 7, 8, 9, 10, 11, 12, 13, 14, 15, 16, 17, 18, 19, 20, 21, 22, or 23;

R′¹, R″¹, R′² and R″² are at each occurrence individually and independently hydrogen, halogen, alkyl, alkoxy, or hydroxyl;

X¹ is each occurrence individually and independently —O—, —S—, —S—S—, —NH—, —(C═O)—, —NH(C═O)—, (C═O)NH— —O(C═O)—, —(C═O)O—, —S(C═O)—, —(C═)S—, —SO—, —SO₂, —NHSO₂—, —SO₂NH—, —(CH2CH2O)_{q}—, —(CH₂)ᵣ—, a disubstituted carbocyclyl, a di-substituted aryl, a disubstituted heterocyclyl, or absent;

X² and Y are at each occurrence individually and independently —O—, —S—, —S—S—, —NH—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH2CH2O)_{q}—, —(CH₂)ᵣ—, a disubstituted carbocyclyl, a di-substituted aryl, a disubstituted heterocyclyl, or absent;

q can be 1 to 1000;

r can be 1 to 22;

G is an tracer, a drug, an antibiotic, an azide group, or other molecule of interest;

R1, R2, R3, and R4, are each individually and independently —(C═O)OCH₂(C═O)alkyl, —O(C═O)OCH₂(C═O)alkyl, hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6;

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R7 is —(C═O)OCH₂O(C═O)alkyl, hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R7 is optionally substituted with one or more, the same or different, R13;

R13 is hydrogen, alkyl, halogen, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl optionally substituted with one or more, the same or different, R14;

R14 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, E is —(CR′R″X)ₘ—(CR′R″Y)ₙ— wherein

m is 1, 2, 3, 4, 5, 6, 7, 8, 9, 10, 11, 12, 13, 14, 15, 16, 17, 18, 19, 20, 21, 22, 23, or 24;

p is 0, 1, 2, 3, 4, 5, 6, 7, 8, 9, 10, 11, 12, 13, 14, 15, 16, 17, 18, 19, 20, 21, 22, or 23;

In certain embodiments, E is —(CH₂)ₘ— wherein m is is 1, 2, or 3.

R′ and R″ are at each occurrence individually and independently hydrogen, alkyl, halogen, or hydroxyl;

X and Y are at each occurrence individually and independently —O—, —S—, —S—S—, —NH—, —(C═O)—, —NH(═O)—, (C═O)NH— —O(C═O)—, —(O═O)O—, —S(C═O)—, —(C═O)S—, —SO—, —SO₂, —NHSO₂—, —SO₂NH—, —(CH₂CH₂O)_{q}—, —(CH₂)ᵣ—, a disubstituted carbocyclyl, a di-substituted aryl, a disubstituted heterocyclyl, or absent;

q can be 1 to 1000; and

r can be 1 to 22.

In certain embodiments, the disclosure relates to compounds of formula I with formula IH,

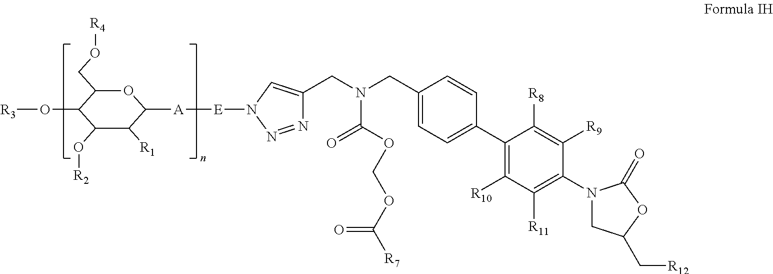

or salts thereof wherein,

n is 2, 3, 4, 5, 6, 7, 8, 9, 10, 11, 12, 13, 14, or 15;

A is O or S at each occurrence provided A must be S at least once or all of A are S;

E is a linking group;

R1, R2, R3, and R4, are each individually and independently independently —(C═O)OCH₂(C═O)alkyl, —O(C═O)OCH₂(C═O)alkyl, hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6;

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, methylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R7 is hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R7 is optionally substituted with one or more, the same or different, R13;

R8, R9, R10, and R11 are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R8, R9, R10, and R11 are optionally substituted with one or more, the same or different, R13;

R12 is acetylamino, hydrogen, alkyl, halogen, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl optionally substituted with one or more, the same or different, R13;

R13 is hydrogen, alkyl, halogen, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl optionally substituted with one or more, the same or different, R14;

R14 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, E is —(CR′R″X)ₘ—(CR′R″Y)ₚ— wherein

m is 1, 2, 3, 4, 5, 6, 7, 8, 9, 10, 11, 12, 13, 14, 15, 16, 17, 18, 19, 20, 21, 22, 23, or 24;

p is 0, 1, 2, 3, 4, 5, 6, 7, 8, 9, 10, 11, 12, 13, 14, 15, 16, 17, 18, 19, 20, 21, 22, or 23;

In certain embodiments, E is —(CH₂)ₘ— wherein m is is 1, 2, or 3.

R′ and R″ are at each occurrence individually and independently hydrogen, alkyl, halogen, or hydroxyl;

X and Y are at each occurrence individually and independently —O—, —S—, —S—S—, —NH—, —(C═O)—, —NH(C═O)—, (C═O)NH— —O(C═O)—, —(C═O)O—, —S(C═O)—, —(C═O)S—, —SO—, —SO₂, —NHSO₂—, —SO₂NH—, —(CH₂CH₂O)_{q}—, —(CH₂)ᵣ—, a disubstituted carbocyclyl, a di-substituted aryl, a disubstituted heterocyclyl, or absent;

q can be 1 to 1000; and

r can be 1 to 22.

In certain embodiments, the disclosure relates to compounds of formula I with formula IK,

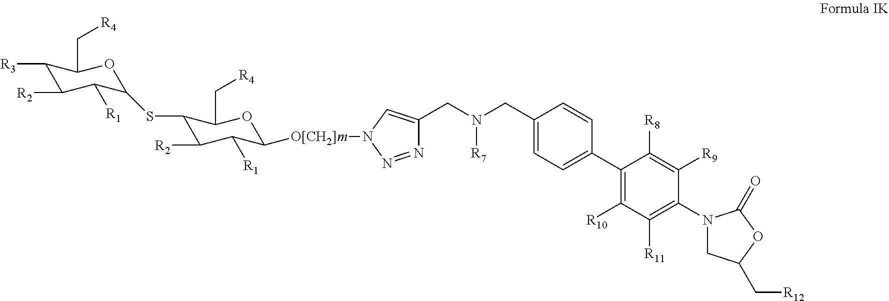

or salts thereof wherein,

m is 2, 3, or 4;

R1, R2, R3, and R4, are each individually and independently —O(C═O)OCH₂O(C═O)alkyl, hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6;

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, methylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R7 is —(C═O)OCH₂O(C═O)alkyl, hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R7 is optionally substituted with one or more, the same or different, R13;

R8, R9, R10, and R11 are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R8, R9, R10, and R11 are optionally substituted with one or more, the same or different, R13;

R12 is acetylamino, hydrogen, alkyl, halogen, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl optionally substituted with one or more, the same or different, R13;

R13 is hydrogen, alkyl, halogen, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl optionally substituted with one or more, the same or different, R14;

R14 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the disclosure relates to compounds of formula I with formula IL,

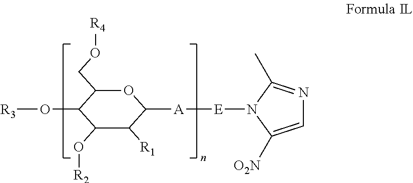

or salts thereof wherein,

n is 2, 3, 4, 5, 6, 7, 8, 9, 10, 11, 12, 13, 14, or 15;

A is O or S at each occurrence provided A must be S at least once or all of A are S;

E is a linking group; for example a formula:

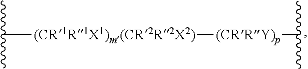

wherein the symbol [symbol] represents the point of attachment to A and imidazole;

m′ is 0, 1, 2, 3, 4, 5, 6, 7, 8, 9, 10, 11, 12, 13, 14, 15, 16, 17, 18, 19, 20, 21, 22, or 23;

p is 0, 1, 2, 3, 4, 5, 6, 7, 8, 9, 10, 110, 12, 13, 14, 15, 16, 17, 18, 19, 20, 21, 22, or 23;

R′¹, R″¹, R′² and R″² are at each occurrence individually and independently hydrogen, halogen, alkyl, alkoxy, or hydroxyl;

X¹ is each occurrence individually and independently —O—, —S—, —S—S—, —NH—, —(C═O)—, —NH(C═O)—, (C═O)NH—, —O(C═O)—, —(C═O)O—, —S(C═O)—, —(C═O)S—, —SO—, —SO₂, —NHSO₂—, —SO₂NH—, —(CH2CH2O)_{q}—, —(CH₂)ᵣ—, a disubstituted carbocyclyl, a di-substituted aryl, a disubstituted heterocyclyl, or absent;

X² and Y are at each occurrence individually and independently —O—, —S—, —S—S—, —NH—, —(C═O)—, —NH(C═O)—, (C═O)NH—, —O(C═O)—, —(C═O)O—, —S(C═O)—, —(C═O)S—, —SO—, —SO₂, —NHSO₂—, —SO₂NH—, —(CH2CH2O)_{q}—, —(CH₂)—, a disubstituted carbocyclyl, a di-substituted aryl, a disubstituted heterocyclyl, or absent;

q can be 1 to 1000;

r can be 1 to 22;

R1, R2, R3, and R4, are each individually and independently —(C═O)OCH₂O(C═O)alkyl, —O(C═O)OCH₂O(C═O)alkyl, hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6; and

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, E is —(CR′R″X)ₘ—(CR′R″Y)ₚ— wherein

m is 1, 2, 3, 4, 5, 6, 7, 8, 9, 10, 11, 12, 13, 14, 15, 16, 17, 18, 19, 20, 21, 22, 23, or 24;

p is 0, 1, 2, 3, 4, 5, 6, 7, 8, 9, 10, 11, 12, 13, 14, 15, 16, 17, 18, 19, 20, 21, 22, or 23;

In certain embodiments, E is —(CH₂)ₘ— wherein m is is 1, 2, or 3.

R′ and R″ are at each occurrence individually and independently hydrogen, alkyl, halogen, or hydroxyl;

X and Y are at each occurrence individually and independently —O—, —S—, —S—S—, —NH—, —(C═O)—, —NH(C═O)—, (C═O)NH—, —O(C═O)—, —(C═O)O—, —S(C═O)—, —(C═O)S—, —SO—, —SO₂, —NHSO₂—, —SO₂NH—, —(CH₂CH₂O)_{q}—, —(CH₂)ᵣ—, a disubstituted carbocyclyl, a di-substituted aryl, a disubstituted heterocyclyl, or absent;

q can be 1 to 1000; and

r can be 1 to 22.

In certain embodiments, the disclosure relates to compounds of formula I with formula IK,

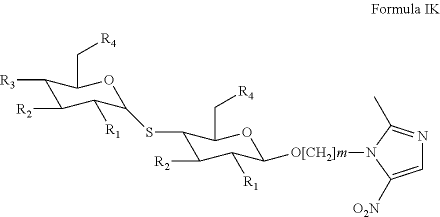

or salts thereof wherein,

m is 2, 3, or 4;

R1, R2, R3, and R4, are each individually and independently —O(C═O)OCH₂O(C═O)alkyl, hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R1, R2, R3, and R4 are optionally substituted with one or more, the same or different, R5;

R5 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R5 is optionally substituted with one or more, the same or different, R6; and

R6 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the disclosure relates to a composition comprising a compound of formula II:

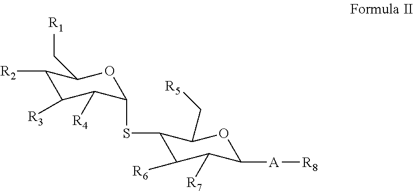

or derivatives thereof wherein,

A is O, NH, S or a direct bond to R8;

R1, R2, R3, R4, R5, R6, and R7 are, the same or different, hydrogen, hydroxy, mercapto, halogen, amino, alkyl, alkoxy, tracer, ¹⁸F optionally substituted with a protecting group or optionally substituted with one or more, the same or different, R9;

R8 is E-G, wherein E is a linking group; G is an tracer, a drug, an antibiotic, an azide group, or other molecule of interest; or

R8 is a protecting group, hydrogen, alkyl, halogen, nitro, cyano, hydroxy, amino, mercapto, formyl, carboxy, alkanoyl, carbamoyl, alkoxy, alkylthio, alkyl amino, (alkyl)₂amino, alkylsulfinyl, alkylsulfonyl, arylsulfonyl, carbocyclyl, aryl, or heterocyclyl, wherein R8 is optionally substituted with one or more, the same or different, R9;

R9 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R9 is optionally substituted with one or more, the same or different, R10;

R10 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R10 is optionally substituted with one or more, the same or different, R11;

R11 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R11 is optionally substituted with one or more, the same or different, R12;

R12 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R12 is optionally substituted with one or more, the same or different, R13; and

R13 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl,

N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, R1, R2, R3, R4, R5, R6, and R7 are, the same or different —O(C═O)OCH₂O(C═O)alkyl, hydrogen, hydroxy, mercapto, halogen, amino, alkyl, alkoxy, tracer, ¹⁸F optionally substituted with a protecting group or optionally substituted with one or more, the same or different, R9.

In certain embodiments, the disclosure relates to compounds of formula II with formula IIA,

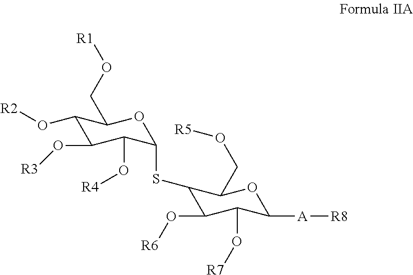

or derivatives thereof wherein,

A is O, NH, S or a direct bond to R8;

R1, R2, R3, R4, R5, R6, and R7 are, the same or different, hydrogen or a protecting group;

R8 is E-G, wherein E is a linking group; G is an tracer, a drug, an antibiotic, an azide group, or other molecule of interest; or

R8 is a protecting group, hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R8 is optionally substituted with one or more, the same or different, R9;

R9 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R9 is optionally substituted with one or more, the same or different, R10;

R10 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R10 is optionally substituted with one or more, the same or different, R11;

R11 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R11 is optionally substituted with one or more, the same or different, R12;

R12 is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R12 is optionally substituted with one or more, the same or different, R13; and

R13 is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl,

N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

R1, R2, R3, R4, R5, R6, and R7 are, the same or different, hydrogen or —(C═O)OCH₂(C═O)alkyl.

### Pharmaceutical Formulations

Within certain embodiments, the disclosure contemplates compounds and conjugates disclosed herein in pharmaceutical composition, optionally as a pharmaceutically acceptable salt, in combination with a pharmaceutically acceptable excipient. Pharmaceutical compositions of the compounds of this application, or derivatives thereof, can be formulated as solutions or lyophilized powders for parenteral administration. Powders can be reconstituted by addition of a suitable diluent or other pharmaceutically acceptable carrier prior to use. The liquid formulation is generally a buffered, isotonic aqueous solution. Examples of suitable diluents are normal isotonic saline solution, 5% dextrose in water or buffered sodium or ammonium acetate solution. Such formulations are especially suitable for parenteral administration but can also be used for oral administration. Excipients, such as polyvinylpyrrolidinone, gelatin, hydroxycellulose, acacia, polyethylene glycol, mannitol, sodium chloride or sodium citrate, can also be added.

Alternatively, these compounds can be encapsulated, tableted, or prepared in an emulsion or syrup for oral administration. Pharmaceutically acceptable solid or liquid carriers can be added to enhance or stabilize the composition, or to facilitate preparation of the composition. Liquid carriers include syrup, peanut oil, olive oil, glycerin, saline, alcohols or water. Solid carriers include starch, lactose, calcium sulfate, dihydrate, terra alba, magnesium stearate or stearic acid, talc, pectin, acacia, agar or gelatin. The carrier can also include a sustained release material such as glyceryl monostearate or glyceryl distearate, alone or with a wax. The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation can be in the form of a syrup, elixir, emulsion, or an aqueous or non-aqueous suspension. Such a liquid formulation can be administered directly p.o. or filled into a soft gelatin capsule.

The pharmaceutical compositions of the application can be in the form of a sterile injectable preparation. Formulations suitable for parenteral administration include aqueous and non-aqueous isotonic sterile injection solutions which can contain antioxidants, buffers, bacteriostats and solutes which render the formulation isotonic with the blood of the intended recipient; and aqueous and non-aqueous sterile suspensions which can include suspending agents and thickening agents.

In some cases, protective groups can be introduced and finally removed. Certain “protective groups” such as an N-acetyl group, can be incorporated and remain as part of the desired compound. Suitable protective groups for amino, hydroxy and carboxy groups are described in Greene et al., Protective Groups in Organic Synthesis, Second Edition, John Wiley and Sons, New York, 1991. Standard organic chemical reactions can be achieved by using a number of different reagents, for examples, as described in Larock: Comprehensive Organic Transformations, VCH Publishers, New York, 1989.

Radio-labeling a small molecule, such as a compound of the present application, usually involves displacement of a suitably activated precursor with a radioactive moiety in a compatible reaction media. In the case of ¹⁸F-labeling, the [¹⁸F]fluoride attachment to the precursor occurs via nucleophilic substitution of a leaving group, such as mesylate, tosylate, bromide, iodide or diazonium salt, or nitro group. Depending on the compound, the preparation of a radio-labeled compound generally consists of at least two steps. The first step involves the preparation of radiolabeling precursor, in which various functional groups have been appropriately protected and a proper leaving group has been incorporated. The second sequence then involves the radio-labeling, and removal of the protecting group as known in the art

### Terms

As used herein, “alkyl” means a noncyclic straight chain or branched, unsaturated or saturated hydrocarbon such as those containing from 1 to 10 carbon atoms, while the term “lower alkyl” or “C1-4 alkyl” has the same meaning as alkyl but contains from 1 to 4 carbon atoms. The term “higher alkyl” has the same meaning as alkyl but contains from 7 to 20 carbon atoms. Representative saturated straight chain alkyls include methyl, ethyl, n-propyl, n-butyl, n-pentyl, n hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles”or heterocarbocyclyl” groups are carbocycles which contain from 1 to 4 heteroatoms independently selected from nitrogen, oxygen and sulfur which can be saturated or unsaturated (but not aromatic), monocyclic or polycyclic, and wherein the nitrogen and sulfur heteroatoms can be optionally oxidized, and the nitrogen heteroatom can be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimnidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems can, but are not required to, contain one or more non-aromatic rings, as long as one of the rings is aromatic.

As used herein, “heteroaryl” refers an aromatic heterocarbocycle having 1 to 4 heteroatoms selected from nitrogen, oxygen and sulfur, and containing at least 1 carbon atom, including both mono- and polycyclic ring systems. Polycyclic ring systems can, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems can be aromatic, non-aromatic or mixtures of aromatic and non-aromatic rings. Heterocycle includes heterocarbocycles, heteroaryls, and the like.

“Alkylthio” refers to an alkyl group as defined above attached through a sulfur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above attached through an amino bridge. An example of an alkylamino is methylamino, (i.e., —NH—CH₃).

“Alkanoyl” refers to an alkyl as defined above attached through a carbonyl bride (i.e., —(C═O)alkyl).

“Alkylsulfonyl” refers to an alkyl as defined above attached through a sulfonyl bridge (i.e., —S(═O)2alkyl) such as mesyl and the like, and “Arylsulfonyl” refers to an aryl attached through a sulfonyl bridge (i.e., —S(═O)₂aryl).

“Alkylsulfinyl” refers to an alkyl as defined above attached through a sulfinyl bridge (i.e. —S(═O)alkyl).

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent can be administered prior to, together with, or after the additional treatment, or a combination thereof.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers any animal, preferably a human patient, livestock, rodent, monkey or domestic pet.

As used herein, the term “derivative” refers to a structurally similar compound that retains sufficient functional attributes of the identified analog. The derivative can be structurally similar because it is lacking one or more atoms, substituted, a salt, in different hydration/oxidation states, or because one or more atoms within the molecule are switched, such as, but not limited to, replacing an oxygen atom with a sulfur or nitrogen and hydrogen or replacing an amino group with a hydroxyl group or vice versa. The derivative can be a prodrug. Derivatives can be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

As used herein, the term “saccharide” refers to sugars or sugar derivatives, polyhydroxylated aldehydes and ketones, e.g., with an empirical formula that approximates Cm(H2O)n, i.e., wherein m and n are the same or about the same. Contemplated saccharides include, e.g., maltose, isomaltose, and lactose with an empirical formula of C12H22O11. The term is intended to encompass sugar monomers, oligomers, and polymers. The terms oligosaccharide and polysaccharide are used interchangeably, and these saccharides typically contain between two and ten monosaccharide units, or greater than ten monosaccharide units. In certain embodiments of the disclosure, the saccharide is a dextrin, maltodextrin, or cyclodextrin. Dextrins are mixtures of polymers of D-glucose units linked by α-(1→4) or α-(1→6) glycosidic bonds. Maltodextrin consists of D-glucose units connected in chains of variable length. The glucose units are primarily linked with α(1→4) glycosidic bonds. Maltodextrin is typically composed of a mixture of chains that vary from three to nineteen glucose units long. Maltose is a disaccharide formed from two units of glucose joined with an α(1→4) bond. Isomaltose has two glucose molecules linked through an α(1→6) bond. In certain embodiments, the disclosure contemplates cyclic and non-cyclic polysaccharides. Typical cyclodextrins contain a number of glucose monomers ranging from six to eight units in a ring, such as alpha cyclodextrin; a six membered sugar ring molecule; beta cyclodextrin, a seven sugar ring molecule; and gamma cyclodextrin, an eight sugar ring molecule.

The term “thiomaltose” and “thiomaltose” analogs refers to maltose, saccharides, or polysaccharides, wherein one or more to the sugar units are connect by a bridging thiol. In certain embodiments, the sugar units are glucose bridged by thiol through a 1→4 and or 1→6 bond.

As used herein, the term “conjugate” or “conjugated,” and the like refer to molecular entities being linked together through covalent bonds. Conjugation can be accomplished by directly coupling the two molecular entities, e.g., creating an ester or amide from a hydroxyl group, amino group, and a carboxylic acid. Conjugation car be accomplished by indirectly coupling the two molecular entities, e.g., instituting a linking group such as a polyethylene glycol. Conjugation can be accomplished by modifying the molecular entities with chemical groups that react with one another, e.g., alkyne-functionalized entity with an azide-functionalized entity or the reduction of thiol groups on individual entities to form a disulfide bond.

“Positron emission tomography (PET) refers to an imaging technique that produces a three-dimensional image by detecting pairs of gamma rays emitted indirectly by a positron-emitting radionuclide tracer. Three-dimensional images of tracer concentration within the area are then constructed by computer analysis. A radioactive tracer is administered to a subject e.g., into blood circulation. Typically there is a waiting period while tracer becomes concentrated in areas of interest; then the subject is placed in the imaging scanner. As the radioisotope undergoes positron emission decay, it emits a positron, an antiparticle of the electron with opposite charge, until it decelerates to a point where it can interact with an electron, producing a pair of (gamma) photons moving in approximately opposite directions. These are detected in the scanning device. The technique typically utilizes simultaneous or coincident detection of the pair of photons moving in approximately opposite direction (the scanner typically has a built-in slight direction-error tolerance). Photons that do not arrive in pairs (i.e. within a timing-window) are typically ignored. One typically localizes the source of the photons along a straight line of coincidence (also called the line of response, or LOR). This data is used to generate an image.

The term “radionuclide” or “radioactive isotope” refers to isotopes exhibiting radioactive decay (i.e., emitting positrons) and radiolabeling agents comprising a radioactive isotope (e.g., [11C]methane, [¹¹C]carbon monoxide, [¹¹C]carbon dioxide, [¹¹C]phosgene, [¹¹C]urea, [¹¹C]cyanogen bromide, as well as various acid chlorides, carboxylic acids, alcohols, aldehydes and ketones containing carbon-11). Such isotopes are also referred to in the art as radioisotopes or radionuclides. Radioactive isotopes are named herein using various commonly used combinations of the name or symbol of the element and its mass number (e.g., ¹⁸F, F-18, or fluorine-18). Exemplary radioactive isotopes include 1-124, F-18 fluoride, C-11, N-13, and O-15, which have half-lives of 4.2 days, 110 minutes, 20 minutes, 10 minutes and 2 minutes, respectively. The radioactive isotope is preferably dissolved in an organic solvent, such as a polar aprotic solvent. Preferably, the radioactive isotopes used in the present method include F-18, C-11, I-123, I-124, I-127, I-131, Br-76, Cu-64, Tc-99m, Y-90, Ga-67, Cr-51, Ir-192, Mo-99, Sm-153 and Tl-201. Other radioactive isotopes that can be employed include: As-72, As-74, Br-75, Co-55, Cu-61, Cu-67, Ga-68, Ge-68, I-125, I-132, In-111, Mn-52, Pb-203 and Ru-97.

Other methods of preparing radiolabeled ligands are well known in the art. Example of such methods are disclosed in, for example: 1) Jewett, D. M. (1992) A Simple Synthesis of [¹¹C]Methyl Triflate Appl. Radiat. Isot. 43, 1383-1385; 2) Crouzel, C. Langstrom, B., Pike, V. W., and Coenen, H. H. (1987) Recommendations for a practical production of [¹¹C]methyl iodide Appl. Radiat. Isot. Int. J. Appl. Instrum. Part A 38, 601-603; Dannals, R. F., Ravert, H. T.; 3) Wilson, A. A. (1990) Radiochemistry of Tracers for Neurotransmitter Receptor Studies. In: Quantitative Imaging: Neuroreceptors, Neurotransmitters, and Enzymes. (Edited by Frost), J. J. Wagner Jr., H. N. pp. 19-35, Raven Press, New York; 4) Jewett, D. M., Manger, T. J., and Watkins, G. L. (1991) Captive Solvent Methods for Fast Simple Carbon-11 Radioalkylations. In: New Trends in Radiopharmaceutical Synthesis, Quality Assurance and Regulatory Control (Edited by Emran, A. M.) pp. 387-391. Plenum Press, New York; 5) Marazano, C., Maziere, M., Berger, G., and Comar, D. (1977) Synthesis of methyl iodide-.sup. 11C and formaldehyde-11C. Appl. Radiat. Isot. 28, 49-52; 6) Watkins, G., Jewett, D., Mulholland, G., Kitbourn, M., and Toorongian, S. (1988) A Captive Solvent Method for Rapid N-[¹¹C]Methylation of Secondary Amides Application to the Benzodiazepine, 4′-Chlorodiazepam (RO5-4864) Appl. Radiat. Isot. 39, 441-444; and 7) Wilson, A. A., DaSilva, J. N., and Houle, S. (1996) In vivo evaluation of [¹¹C] and [¹⁵F]-labeled cocaine analogs as potential dopamine transporter ligands for positron emission tomography Nucl. Med. Biol. 23, 141-146. The subject matter of all references cited herein are incorporated herein by reference in their entirety.

As used herein, a “linker” refers to any molecular configuration that joins molecular moieties. It includes molecules with covalent and non-covalent interactions. A prefer linker is a polymer, i.e., molecule with repeated linking moieties. The linked moieties can be identical in structure or vary, e.g., copolymers. Linking polymers include, but are not limited to, biological polymers, polyethylene glycols, hydrocarbon chains, alkylacrylates, alkylacrylamides, amides, esters, polypeptides, and derivatives thereof.

A “protecting group” refers to those moieties that are introduced into a molecule by chemical modification of a functional group in order to obtain chemoselectivity in a subsequent chemical reaction or to facilitate purification. Protecting groups can be categorized by the reaction conditions and/or reagents that are used to remove them such as acid labile protecting groups, base labile protecting groups and hydrogenation removable protecting groups. For example, acid labile protecting groups, such as tBu or Boc, typically decompose when exposed to strong acidic conditions providing a hydrogen substituent in place of tBu or Boc protecting group. Acetyl esters and thioesters of alcohols and thiols are examples of base labile protecting groups. Additional examples of protecting groups include, but are not limited to, 4-methoxy-2,3,6-trimethylphenyl)sulfonyl (Mtr), 2,2,5,7,8-pentamethyl-chroman-6-sulphonyl (Pmc), tosyl (Tos), mesitylenesulfonyl (Mts), 4,4′-dimethoxybenzhydyl (Mbh), 2,4,6-trimethoxybenzyl (Tmob), tripheylmethyl (Trt), 9-fluorenylmethyloxycarbonyl (fmoc), tert-butyl (tBu), benzyl (Bzl), t-butoxymethyl ether (Bum), (2,4-dinitrophenol) Dnp, benzyloxymethyl (Bom), benzyloxycarbonyl (Z), 2-chloro-benzyloxycarbonyl (CIZ), t-butyloxycarbonyl (Boc), formyl (CHO) or 2-bromobenzyloxycarbonyl (BrZ) and heterocycles such as succinimide, maleimide, and phathalimide. Protecting groups can be in the form of derivatives, e.g., having one or more substituents.

### Examples

The following examples are put forth so as to provide those of ordinary skill in the art with a complete disclosure and description of how to make and use the assay, screening, and therapeutic methods of the disclosure, and are not intended to limit the scope of what the claimed embodiments.

### Synthesis of Thiomaltose-Perylene

In order to test the specificity of thiomaltose for bacteria, a thiomaltose derivative labeled with the fluorescent dye was synthesized, perylene (15), using an azido-thiomaltose (14) as the synthetic intermediate. The procedure used for the synthesis of thiomaltose-perylene (15) is shown in FIG. 2. Azido-thiomaltose (14) was first synthesized by lewis acid promoted glycosylation between the thiomaltose imidate donor (13) and azidopropanol. Thiomaltose-perylene was then synthesized by conjugating the perylene dye onto azidothiomaltose (14) via the click reaction, followed by deprotection of the acetyl protecting groups using lithium hydroxide. Synthetic details for the synthesis of thiomaltose (10), azidothiomaltose (14) and thiomaltose-perylene (15) are provided.

### Synthesis of Thiomaltose (10)

To a stirred solution of α-D-glucopyranose, 4-S-(2,3,4,6-tetra-O-acetyl-β-D-glucopyranosyl)-4-thio-1,2,3,6-tetrabenzoate 9 (1.0 g, 10 mmol) in anhydrous CH₃OH (15 mL) was added NaOCH₃ (100 mg, 20 mmol) under an atmosphere of N₂ at room temperature, and the reaction was stirred at room temperature for 12 hours. The reaction mixture was neutralized by adding acidic DOWEX resins, filtered, and concentrated in vacuo. The resulting residue was re-dissolved in DMF (10 mL), and precipitated by adding ether (100 mL). The precipitated powder was collected and dried in vacuo to afford the product 10 (300 mg, 83%) (α and β mixture). ¹H NMR (D2O, 400 MHz): δ (ppm) 5.01 (d, 3J (H,H)=10.2 Hz, 0.6H), 4.82 (d, 3J (H,H)=4.8 Hz, 1H), 4.33 (d, 3J (H,H)=3.6 Hz, 0.4H), 3.71-3.74 (m, 2H), 3.67-3.63 (m, 3H), 3.5-3.49 (m, 2H), 3.43 (dd, 3J (H,H)=10.2 Hz, 1H), 3.26-3.21 (m, 3H), 3.0-2.98 (m, 1H), 2.83-2.85 (m, 1H). 13C NMR (D2O, 100 MHz): δ (ppm) 95.8, 91.5, 85.2, 85.1, 85.1, 81.2, 80.0, 79.6, 78.5, 77.9, 77.8, 77.0, 76.8, 75.8, 74.3, 74.1, 73.7, 73.1, 70.8, 62.8, 62.7, 62.2, 61.5, 48.1, 47.5. MS (MALDI) m/z Found: 381.79, calculated: C₁₂H₂₂O₁₀SNa [M+Na+] 381.08.

### Synthesis of Azidothiomaltose (14)

To a stirred solution of thiomaltose imidate 13 (0.16 g, 0.2 mmol) and 3-azidopropanol (0.1 g, 1.0 mmol) in dry DCM (5 mL) was added 4 Å M.S. The mixture was stirred under nitrogen at 0° C. for 1 hour. TMSOTf (45 μL, 0.20 mmol) was then added and the mixture was stirred at 0° C. for 2 hour. The reaction was quenched with Et₃N and concentrated in vacuo. The residue was dissolved in EtOAc (20 mL) and washed with water (5 mL×2) and brine (10 mL). The organic phase was dried over Na₂SO₄, filtered and evaporated to dryness in vacuo. The residue was purified by flash column chromatography on silica gel (hexane/EtOAc, 1:1) to afford 14 (92.6 mg, 63%). ¹H NMR (400 MHz, CDCl3): δ (ppm) 5.89 (d, 1H, J=5.8 Hz, 1-H (α thiol linkage)), 5.28-5.21 (m, 2H), 5.06 (1H, m), 4.95 (1H, m), 4.78 (1H, m), 4.65 (1H, m), 4.46 (1H, d, J=8.6 Hz, 1-H (β)), 4.33 (1H, m), 4.25-4.20 (2H, m), 4.11 (1H, m), 3.89 (1H, m), 3.58-3.63 (2H, m), 3.35 (2H, t, J=6.0 Hz, CH2), 2.97 (2H, t, J=6.0 Hz, CH2), 2.12-1.99 (s, 21H, CH3), 1.83 (2H, m, CH2). 13C NMR (100 MHz, CDCl3): δ (ppm) 170.5, 170.4, 170.2, 169.9, 169.8, 169.5, 169.4, 100.3, 82.4, 76.7, 75.7, 72.7, 72.5, 70.3, 70.1, 69.7, 68.6, 67.9, 66.3, 63.6, 61.5, 47.9, 43.7, 28.9, 20.8, 20.7, 20.6, 20.6, 20.5, 20.4. HRMS (MALDI) m/z Found: 758.2089, calculated: 758.2054 for C₂₉H₄₁N₃O₁₇SNa [M+Na]+.

### Synthesis of Thiomaltose-Perylene (15)

To a stirred solution of 14 (15.0 mg, 0.02 mmol) and alkyne functionalized perylene dye (13.0 mg, 0.04 mmol) in DMF (5 mL) was added CuI (0.2 mg, 1.0 μmol) and DIPEA (1.2 mg, 0.01 mmol). The mixture was stirred at room temperature for 12 hours under nitrogen and the solvent was removed in vacuo. The residue was dissolved in DCM (10 mL) and washed with water (5 mL×2) and brine (5 mL). The organic phase was dried over Na₂SO₄, filtered and evaporated to dryness in vacuo. The residue was dissolved in CH₃OH (5 mL), and aqueous LiOH (1.0 M, 2 mL) was added into the reaction mixture under nitrogen. The reaction mixture was stirred at room temperature for 24 hours. The mixture was then neutralized with Dowex 50W resin, filtered and concentrated in vacuo. The residue was purified by HPLC to afford 15 (10.0 mg, 65% in two steps). ¹H-NMR (400 MHz, DMSO-d6): δ (ppm) 7.85-7.76 (m, 3H, Aromatic), 7.72 (d, 1H, J=8.0 Hz, ArH), 7.51 (d, 1H, J=8.0 Hz, ArH), 7.39 (s, 1H, triazole), 7.21 (d, 2H, J=8.0 Hz, ArH), 7.13-7.03 (m, 4H, ArH), 5.96 (d, 1H, J=6.0 Hz, 1-H′ (α thiol linkage), 4.96-4.91 (2H, m), 4.71 (m, 2H, ArCH2O), 4.55 (s, 2H, CH2-C═C), 4.45 (d, 1H, J=8.4 Hz, 1H (β)), 3.78-3.71 (5H, m), 3.69-3.67 (2H, m), 3.43-3.31 (5H, m), 3.22-3.20 (m, 3H), 2.91-2.89 (m, 1H), 1.91 (2H, m). 13C NMR (100 MHz, DMSO-d6): δ (ppm) 143.3, 134.8, 133.1, 132.8, 131.9, 131.7, 131.0, 128.9 128.5, 127.9, 127.9, 126.7, 126.5, 126.1, 123.7, 123.6, 120.1, 120.0, 120.0, 119.5, 103.6 (1-C(β)), 97.8 (1-C(α thiol linkage)), 81.5, 79.1, 77.3, 76.9, 75.7, 73.8, 73.1, 72.8, 70.6, 68.1, 64.3, 62.0, 48.7, 47.9, 28.5. HRMS (MALDI) m/z Found: 784.2543, calculated: 784.2516 for C₃₉H₄₃N₃O₁₁SNa [M+Na]+.

### Thiomaltose has High Specificity for Bacteria Over Mammalian Cells

Thiomaltose-perylene has high specificity for bacteria over mammalian cells. The uptake of thiomaltose-perylene was investigated in E. coli and in Raw 264.7 murine macrophages. A 500 μL suspension of E. coli (O.D=0.6) was incubated with 20 μM thiomaltose-perylene for 2 hours. The bacterial cells were washed in PBS and lysed. The fluorescence intensity of the sample was measured and normalized to the protein content. Similarly, 10⁵ macrophage cells were incubated with 20 μM thiomaltose-perylene for 2 hours and the cells were lysed. The specificity of thiomaltose-perylene for bacteria was determined by comparing the fluorescence intensity in bacteria versus macrophages, normalized to intracellular protein content. FIG. 13 indicates that thiomaltose-perylene has high specificity for bacteria, as the uptake of thiomaltose-perylene was 98 fold higher in bacteria when compared to mammalian cells.

### Thiomaltose is Stable to Maltase

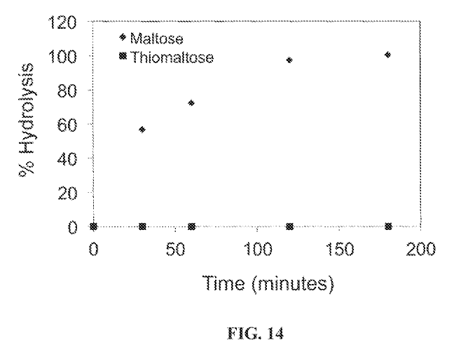

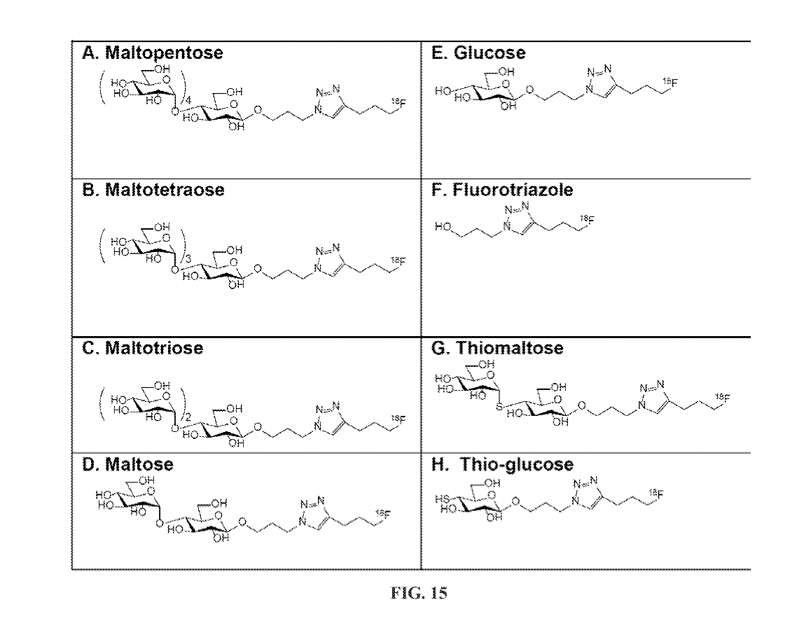

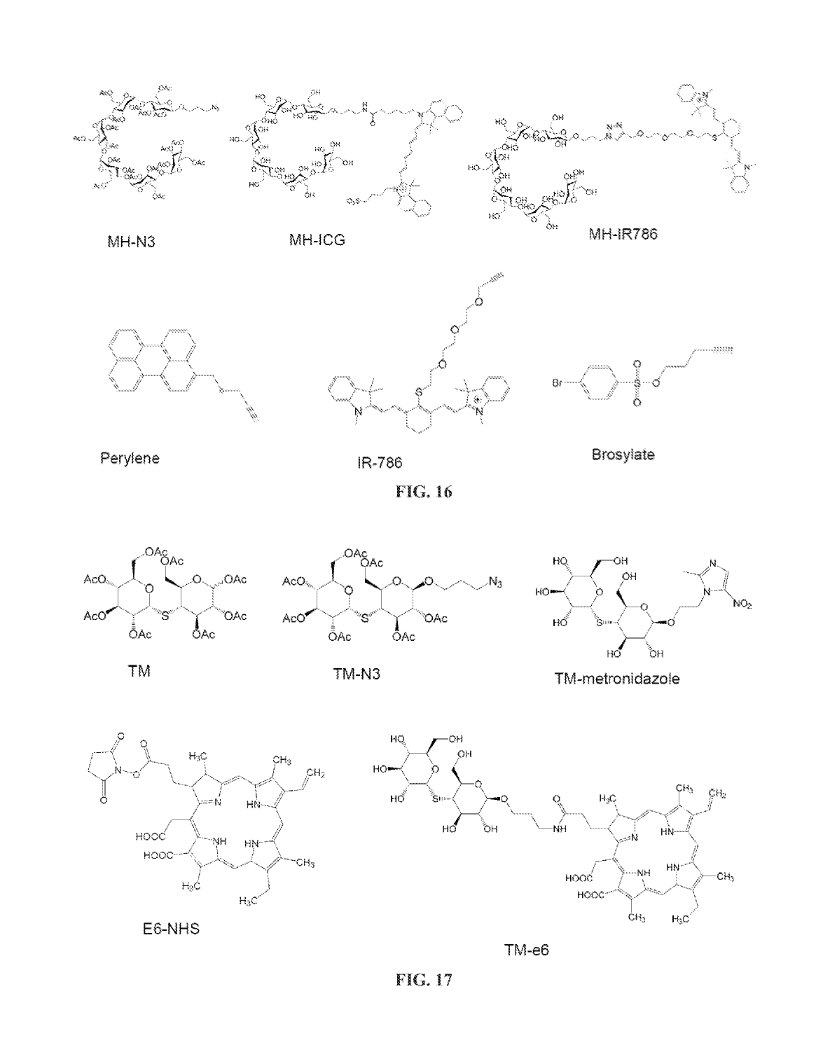

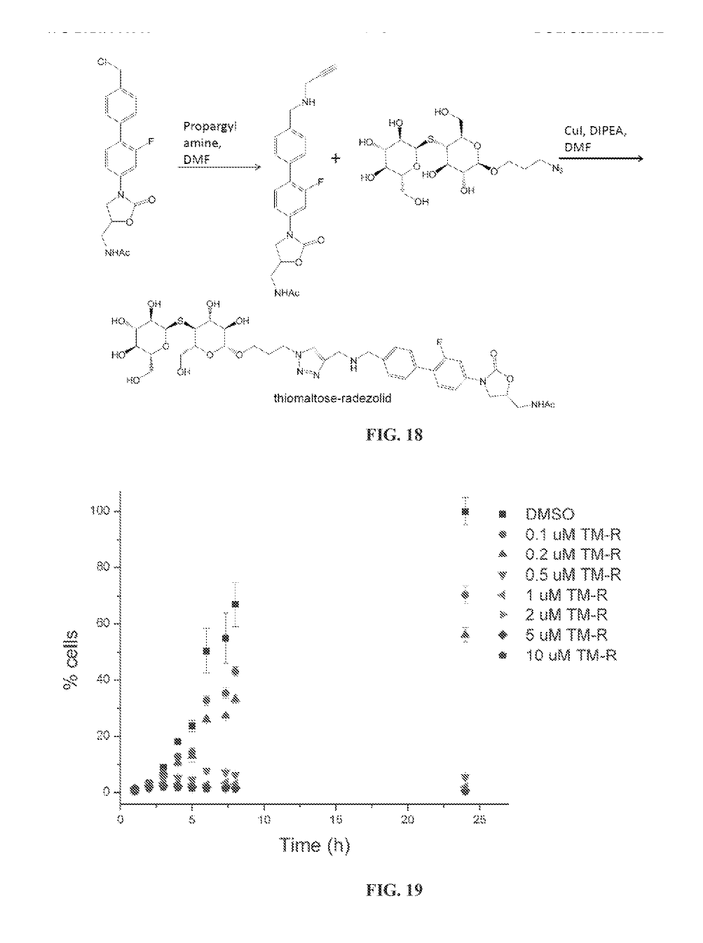

Thiomaltose is stable to maltase hydrolysis. The stability of thiomaltose and maltose was investigated in the presence of maltase. Thiomaltose and maltose were incubated with 10 units of maltase, which is an enzyme that breaks maltose into two glucose molecules. The hydrolysis kinetics of thiomaltose and maltose was determined by quantifying the glucose released from thiomaltose or maltose hydrolysis. FIG. 14 demonstrates that thiomaltose is orders of magnitude more stable to maltase hydrolysis than maltose. For example, the half-life of maltose is approximately 30 minutes, under these conditions, whereas thiomaltose had negligible hydrolysis after 3 hours.

### Synthesis of ¹⁸F-thiomaltose

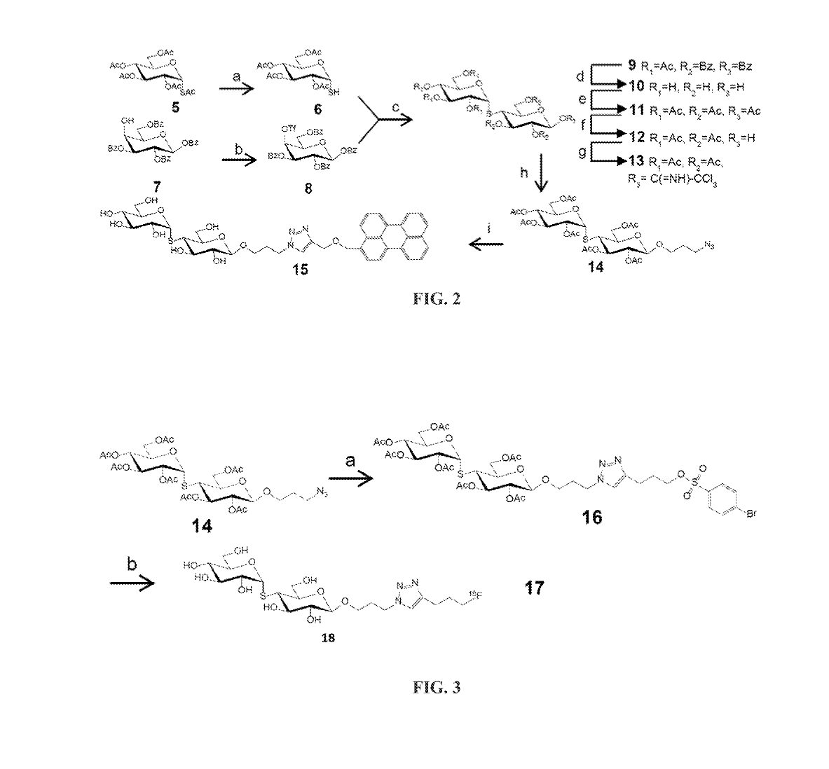

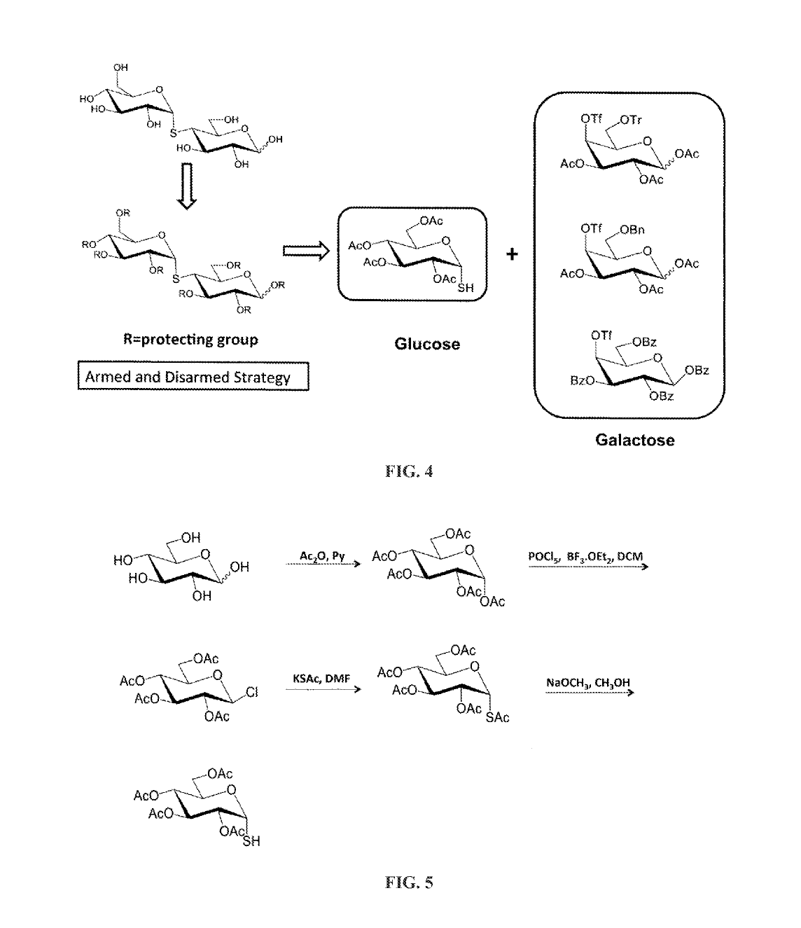

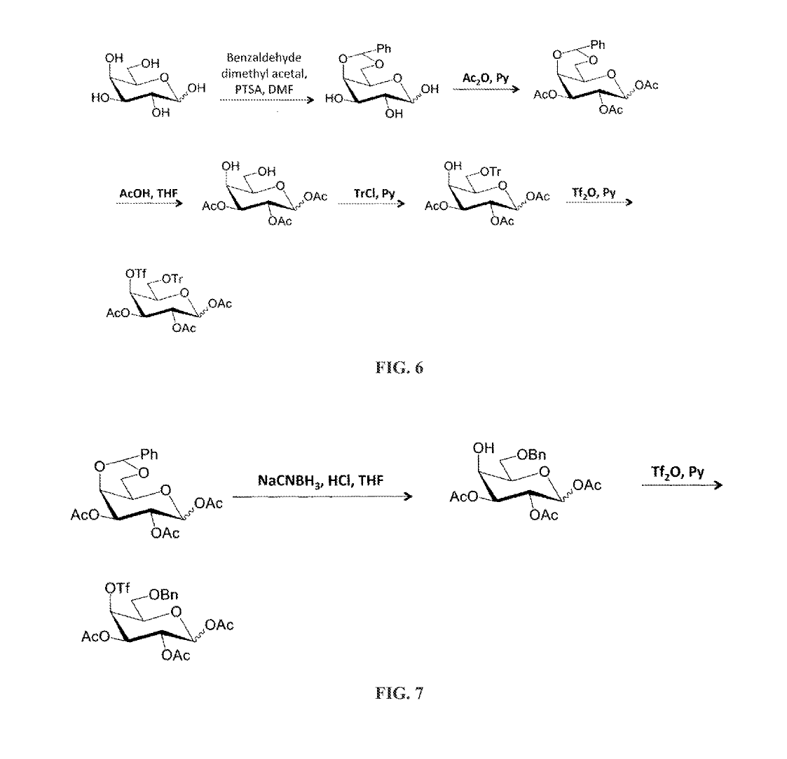

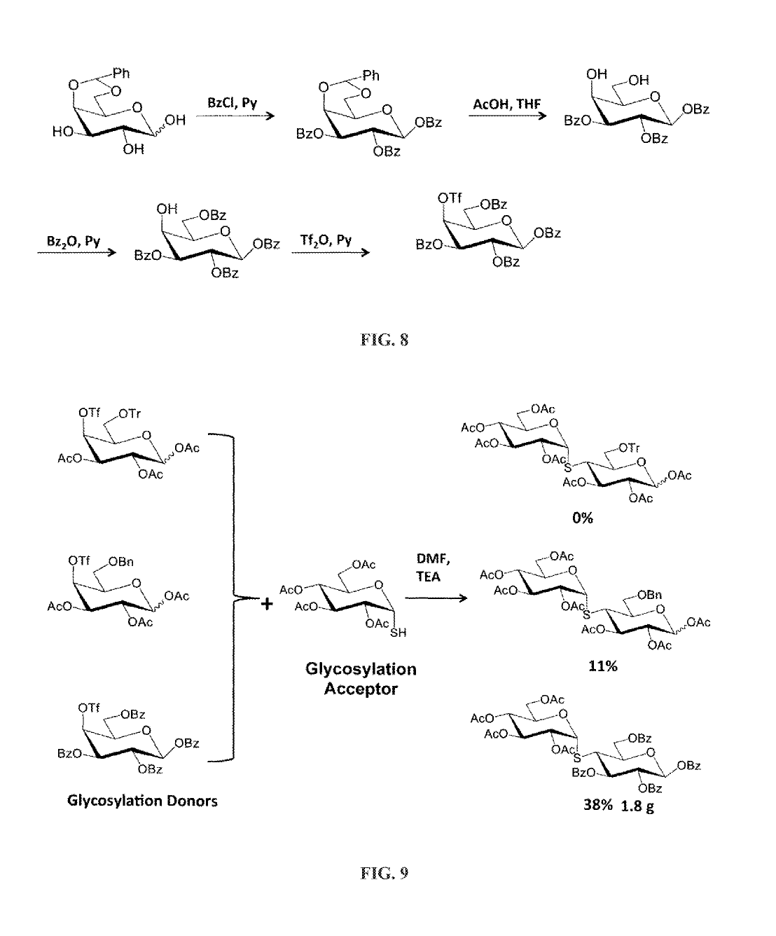

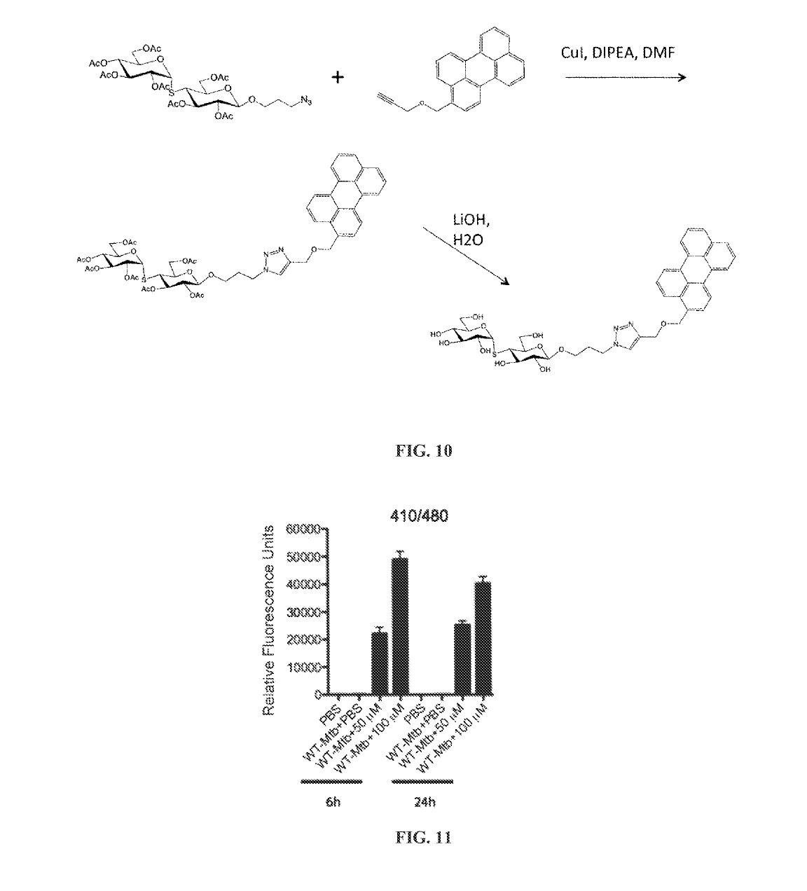

One can synthesize ¹⁸F-thiomaltose (17), following the general procedure shown in FIG. 3. Briefly, one converts azido-thiomaltose (14) into a brosylate precursor (16) via a click reaction with pent-4-yn-1-yl 4-bromobenzenesulfonate (2). One obtains ¹⁸F-thiomaltose (17) by reacting the precursor (16) with K¹⁸F, followed by deprotection of the acetyl protecting groups with aqueous NaOH. One can purify intermediates by flash column chromatography on silica gel and characterized by NMR and HRMS, and one can purify ¹⁸F-thiomaltose by HPLC.

### Synthesis of Thiomaltose-Metronidazole TMM (14)

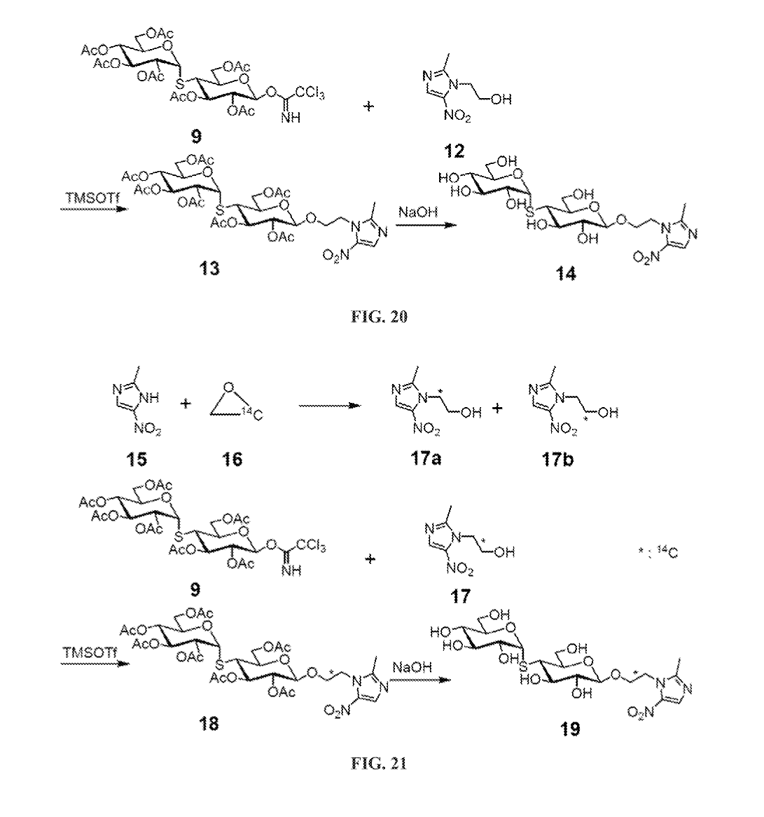

One can conjugate metroinadazole to thiomaltose and generate TMM (See FIG. 20). To a stirred solution of thiomaltose imidate 9 (100 mg, 0.13 mmol) and metronidazole 12 (43 mg, 0.26 mmol) in dry DCM (5 mL) was added 4 Å M.S. The mixture was stirred under nitrogen at 0° C. for 1 hour. TMSOTf (10 μL, 0.044 mmol) was then added and the mixture was stirred at 0° C. for 2 hour. The reaction was quenched with Et3N and concentrated in vacuo. The residue was dissolved in EtOAc (20 mL) and washed with water (5 mL×2) and brine (10 mL). The organic phase was dried over Na2SO4, filtered and evaporated to dryness in vacuo. The residue was purified by flash column chromatography on silica gel (hexane/EtOAc, 1:1) to afford 13 (53 mg, 52%) 1H NMR (400 MHz, CDCl3): δ (ppm) 7.95 (s, 1H), 5.87 (d, 1H), 5.28-5.16 (m, 2H), 5.07 (t, 1H) 4.98-4.94 (m, 1H), 4.74 (t, 1H), 4.64-4.56 (m, 2H), 4.39-4.32 (m, 3H), 4.24-4.08 (m, 6H), 3.86 (t, 1H), 3.59 (t, 1H), 2.95 (t, 1H), 2.46 (s, 3H), 2.14-1.95 (m, 21H). ESI-MS m/z Found: 837.2286, calculated: 806.2284 for C32H44N3O19S [M+H]+.

TMM 14 was synthesized by deprotection of 13 with NaOH. To a stirred solution of 13 (40 mg, 0.005 mmol) in MeOH (2 mL) and water (0.5 mL) was added NaOH (40 mg, 1.0 mmol). The mixture was stirred overnight and purified by HPLC to afford TMM (9.1 mg, 27.6%). 1H NMR (400 MHz, D20): δ (ppm) 8.39 (s, 1H), 5.50 (d, 1H), 4.24-4.15 (m, 3H), 4.04-3.95 (m, 21H) 3.86-3.83 (nm, 4H), 3.74-3.94 (m, 15H), 3.26 (t, 21H), 3.05 (t, 2H), 2.67 (s, 5H), 2.56 (t, 2H). MALDI-MS m/z Found: 514.6, calculated: 512.1544 for C18H30N3O12S [M+H]+.

### TMM is Effective at Killing Giardia

Methods for evaluating EC50 measurements in Giardia were established. Giardia cells were cultured in 96 well microtiter plates in anaerobic BD Bio-Bags (Becton Dickinson). Growth was assayed with a MoxiZ coulter counter (Orflo Technologies). The TMM and metroinadazole stock solutions contained DMSO to improve solubility. Giardia growth in 96 well plates with various volumes of TYDK media 10 and DMSO concentrations were evaluated over several days at 37° C. Cells grew more consistently in higher volumes of media and noted reduced growth in DMSO concentrations above 0.25%. Culture volumes of 180 uL (10,000 cells/mL starting concentration) and the DMSO concentrations at or below 0.1% were used. Growth was evaluated after 48 hours. The 48 hour time point allows several cell cycles of log phase growth, but stops before maximum densities of 1×10⁶ cells/mL are reached TMM has an EC50 of 1.53 μM while metronidazole has an EC50 of 1.7 μM indicating that TMM is at least as good as metronidazole but appears more potent, thus demonstrating that thiomaltose conjugation does not interfere with the efficacy of metronidazole.

### Synthesis of Thiomaltose-Radezolid (TMR) (14)

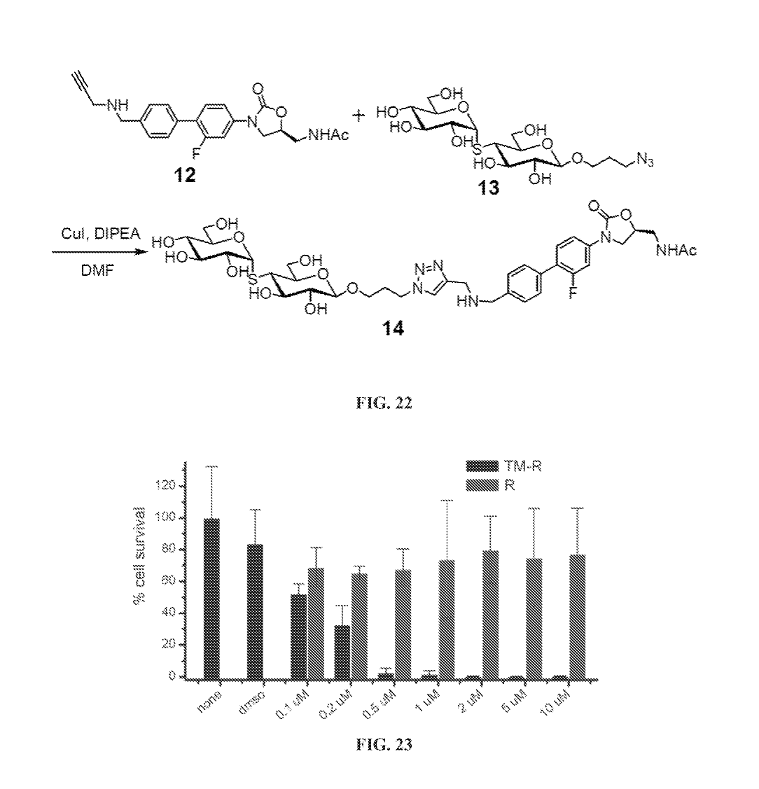

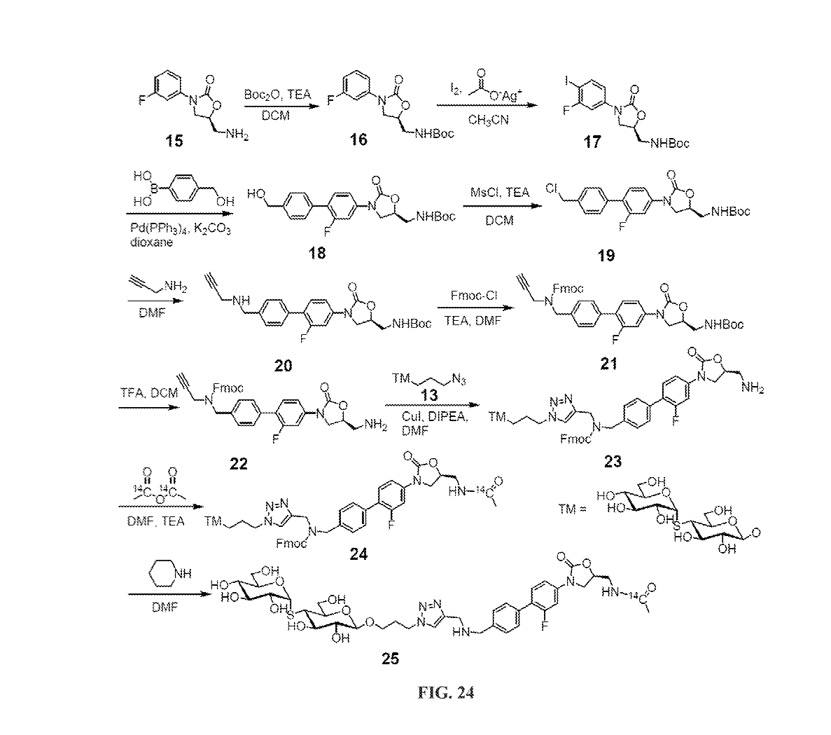

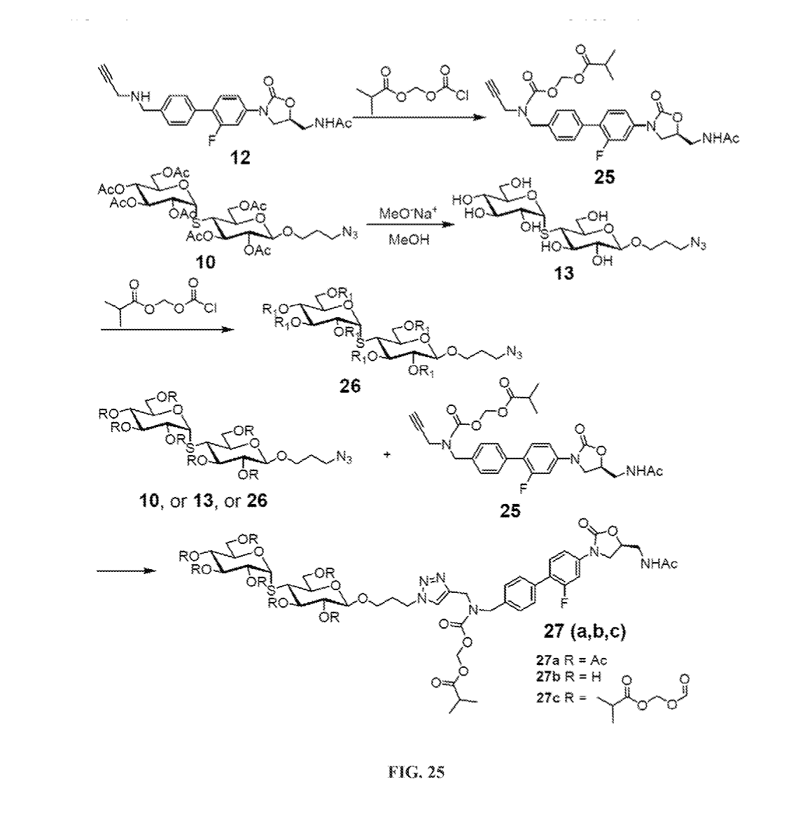

The synthesis of a conjugate of thiomaltose and radezolid (TMR) is illustrated in FIG. 22. The synthesis of TMR is described below. To a stirred solution of 12 (8.0 mg, 0.02 mmol) and 13 (8.8 mg, 0.02 mmol) in DMF (2 mL) was added DIPEA (50 μL) and CuI (0.1 mg, 0.53 μmol), after stirring for 24 h, the solvent was removed in vacuo. The crude product was purified by HPLC to afford thiomaltose-radezolid (TMR, 14) (12.0 mg, 71%). 1H NMR (400 MHz, D2O): δ (ppm) 8.06 (s, 1H), 7.56-7.33 (m, 6H), 7.21 (d, 1H), 5.60 (d, 1H), 4.51 (t, 2H), 4.40 (s, 1H) 4.33-4.20 (m, 4H) 4.11 (t, 1H), 3.92 (d, 2H), 3.85-3.70 (m, 7H), 3.60-3.33 (m, 8H), 3.22-3.11 (m, 2H), 2.84 (s, 2H), 2.70 (t, 1H), 2.18-2.07 (m, 2H), 1.98 (s, 3H), 1.34-1.27 (m, 2H). ESI-MS m/z Found: 837.3122, calculated: 837.3135 for C37H50FN6O13S [M+H]+.

TMR is More Effective at Killing P. aeruginosa than Free Radezolid

The efficacy of radezolid can be increased by conjugating it to thiomaltose. P. aeruginosa (5×10⁸ CFUs) were incubated with various concentrations of either radezolid or TMR and the MIC was determined via absorption measurements. The P. aeruginosa strain used in this experiment was a clinical isolate, resistant to multiple antibiotics, obtained from a blood stream infection. FIG. 23 shows data indicating that TMR is 1-2 orders of magnitude more effective at killing pseudomonas than free radezolid. For example, a 0.5 μM concentration of TMR causes a 95% reduction in P. aeruginosa growth, whereas free radezolid had no efficacy up to a 10 μM concentration. Thus TMR is able to improve the efficacy of radezolid, presumably by enhancing its transport into GNB.

1. A compound having the formula: 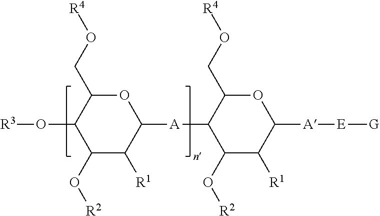
or a salt thereof,
wherein,
R¹, R², R³, and R⁴, are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R¹, R², R³, and R⁴ are optionally substituted with one or more, the same or different, R⁵;
R⁵ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶;
R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, thylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;
n′ is 1, 2, 3, 4, 5, 6, 7, 8, 9, 10, 11, 12, 13, or 14;
A is O or S at each occurrence provided A must be S at least once or all of A are S;
A′ is O or S;
E is a linking group; and
G is a radionuclide, fluorescent molecule, an antibiotic, or an azide group.

2. The compound according to claim 1, wherein E is: 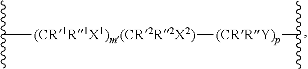
wherein the symbol [symbol] represents the point of attachment to A′ and G;
m′ is 0, 1, 2, 3, 4, 5, 6, 7, 8, 9, 10, 11, 12, 13, 14, 15, 16, 17, 18, 19, 20, 21, 22, or 23;
p is 0, 1, 2, 3, 4, 5, 6, 7, 8, 9, 10, 11, 12, 13, 14, 15, 16, 17, 18, 19, 20, 21, 22, or 23;
R′¹, R″¹, R′² and R″² are at each occurrence independently selected from the group consisting of: hydrogen, halogen, alkyl, alkoxy, and hydroxyl;
X¹, X² and Y are at each occurrence independently selected from the group consisting of: —O—, —S—, —S—S—, —NH—, —(C═O)—, —NH(C═O)—, (C═O)NH—, —O(C═O)—, —(C═O)O—, —S(C═O)—, —(C═O)S—, —SO—, —SO₂, —NHSO₂—, —SO₂NH—, —(CH2CH2O)_{q}—, —(CH₂)ᵣ, a disubstituted carbocyclyl, a di-substituted aryl, and a disubstituted heterocyclyl, or are absent;
q is from 1 to 1000; and
r is from 1 to 22.

3. The compound according to claim 1, wherein A′ is O.

4. The compound according to claim 2, wherein X¹ is in each case absent.

5. The compound according to claim 2, wherein X² comprises a heterocyclic ring.

6. The compound according to claim 2, wherein X² comprises a triazole ring.

7. The compound according to claim 1, wherein R², R³, and R⁴ are in each case hydrogen and R¹ is independently selected from H, OH, and F.

8. The compound according to claim 1, R¹ is independently selected from H, OH, and F.

9. The compound according to claim 1, wherein G is selected from the group consisting of:
—(CH₂)ₓ¹⁸F, wherein x can be 1, 2, 3, 4, 5, 6, 7 or 8, 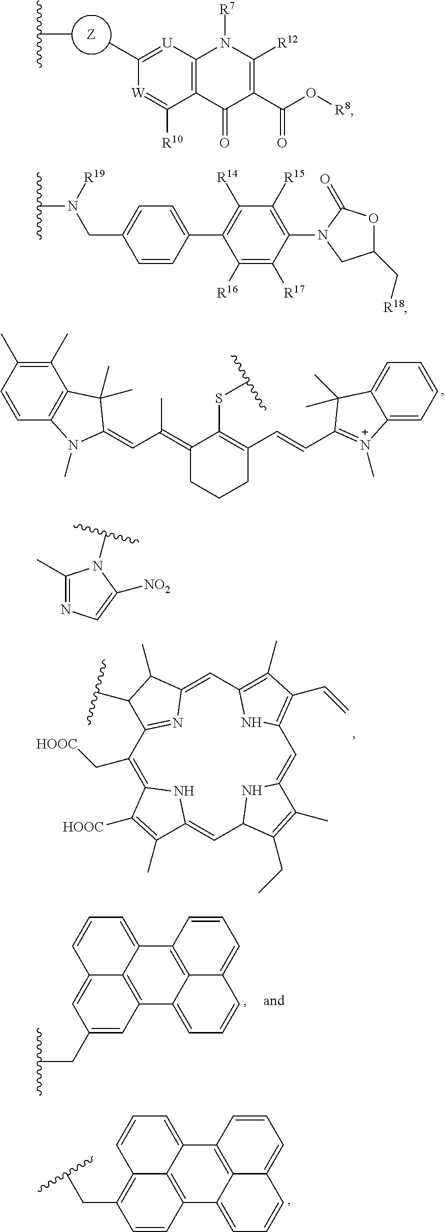
wherein the symbol [symbol] represents the point of attachment to E;
U is N or CR¹¹;
W is N or CR⁹;
Z is a carbocyclic or heterocyclic ring;
R⁷ is alkyl, carbocyclyl, or aryl, wherein R⁷ is optionally substituted with one or more, the same or different R¹³; or R⁷ and R¹¹ form a heterocarbocyclic ring optionally substituted with R¹³;
R⁸ is hydrogen, alkyl or alkanoyl;
R⁹ is a hydrogen or halogen;
R¹⁰ is hydrogen, alkoxy, amino, or alkyl;
R¹¹ is hydrogen, alkoxy, or halogen;
R¹² is hydrogen;
R¹³ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;
R¹⁹ is hydrogen, alkyl, carbocyclyl, or aryl, wherein R⁷ is optionally substituted with one or more, the same or different R¹³;
R¹⁴, R¹⁵, R¹⁶, and R¹⁷ are each individually and independently hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R¹⁴, R¹⁵, R¹⁶, and R¹⁷ are optionally substituted with one or more, the same or different, R²⁰;
R¹⁸ is acetylamino, hydrogen, alkyl, halogen, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl optionally substituted with one or more, the same or different, R¹³; and
R²⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, Nmethylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

10. The compound according to claim 9, wherein Z is selected from the group consisting of: 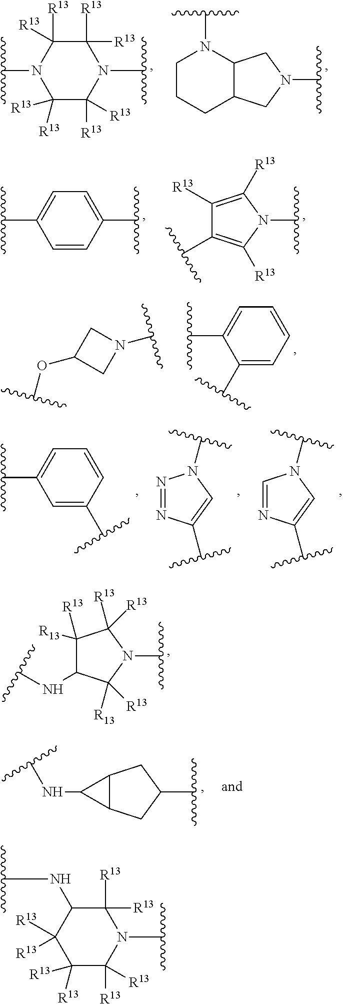
wherein the symbol [symbol] represents the point of attachment to E or to the quinolone fragment and R¹³ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethyl sulfamoyl, N-methyl-N-ethylsulfamoyl, carbocyclyl, aryl, or heterocyclyl.

11. (canceled)

12. The compound according to claim 2, wherein X¹ is in each case absent, R′, R″, R′¹, R″¹, R′², R″² are in each case hydrogen and X² is heterocycle.

13. The compound according to claim 2, wherein X² is:

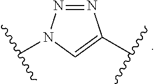 [SMILES: *c1cn(*)nn1]

14. The compound according to claim 2, wherein Y is absent.

15. The compound according to claim 2, wherein n′ is 1, 2, 3, or 4.

16-18. (canceled)

19. The compound according to claim 1, wherein G comprises a radionuclide comprising a positron-emitting radionuclide.

20. (canceled)

21. The compound according to claim 1, wherein G comprises a tracer molecule comprising a fluorescent molecule.

22-25. (canceled)

26. The compound according to claim 1, wherein G comprises an antibiotic.

27-32. (canceled)

33. A method of treating or preventing a bacterial infection comprising administering an effective amount of the compound of claim 26.

34. A compound having the following formula: 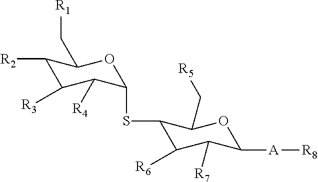
or derivative thereof wherein,
A is O, NH, S or a direct bond to R₈;
R₁, R₂, R₃, R₄, R₅, R₆, and R₇ are independently selected from the group consisting of hydrogen, hydroxy, halogen, alkyl, alkoxy, tracer, and ¹⁸F;
R₈ is E-G, wherein E is a linking group; G is an tracer, a drug, an antibiotic, an azide group, or other molecule of interest; or
R₈ is a protecting group, hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R₈ is optionally substituted with one or more, the same or different, R₉;
R₉ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R₉ is optionally substituted with one or more, the same or different, R₁₀;
R₁₀ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R₁₀ is optionally substituted with one or more, the same or different, R₁₁;
R₁₁ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R₁₁ is optionally substituted with one or more, the same or different, R₁₂;
R₁₂ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R₁₂ is optionally substituted with one or more, the same or different, R₁₃; and
R₁₃ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-Nethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

35. The composition of claim 34 wherein, E is triazole positioned between linking groups, wherein each linking group is independently selected from the group ether, amine, amide, ester, carbonyl, thiol, dithiol, thiolester, aromatic, heteroaromatic, and hydrocarbon groups.
